(19) United States
(12) Patent Application Publication
(10) Pub. No.: US 2009/0214472 A1
(43) Pub. Date: Aug. 27, 2009

(54) Interferon-beta polymer conjugates

(75) Inventors: David Ray Filpula, Piscataway, NJ (US); Karen Yang, Edison, NJ (US); Amartya Basu, East Hanover, NJ (US); Maoliang Wang, East Brunswick, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS INC., BRIDGEWATER (NJ)

(21) Appl. No.: 10/590,421

(22) PCT Filed: Feb. 28, 2005
(86) PCT No.: PCT/US05/06575

§ 371 Date: Sep. 8, 2008

Related U.S. Application Data
(60) Provisional application No. 60/549,109, filed on Mar. 1, 2004.

(51) Int. Cl. A61K 38/21; C07K 1/02

(52) U.S. Cl. 424/85.6; 424/85.4; 530/412

(57) ABSTRACT

Biologically-active, interferon-beta 1b-polymer conjugate compositions are disclosed. The polymer portion is preferably a polyalkylene oxide polymer having a molecular weight of at least about 12 kDa. Methods of making and using the same are also disclosed.

### BACKGROUND OF THE INVENTION

1. Field of the Invention

The present invention is directed to beta interferon-polymer conjugates. In particular, the invention is directed to polymer conjugates of interferon beta 1b and substantially non-antigenic polymers such as PEG.

2. Description of the Related Art

Many proteins or polypeptides are known that hold great promise for use in treating a wide variety of diseases or disorders. Unfortunately, protein therapeutics suffer from a number of drawbacks, including poor solubility in water and body fluids, rapid clearance from the bloodstream after administration, and the potential to elicit an immune response from the treated person or animal. One proposed solution to addressing these drawbacks is to conjugate such proteins or polypeptides to substantially non-antigenic polymers in order to improve circulating life, water solubility and/or to reduce antigenicity. For example, some of the initial concepts of coupling peptides or polypeptides to polyethylene glycol (PEG) and similar water-soluble polymers are disclosed in U.S. Pat. No. 4,179,337, the disclosure of which is incorporated herein by reference.

Interferons, also referred to herein as IFNs, are one class of therapeutic proteins that will benefit from improved circulating life, water solubility and/or reduced antigenicity. Interferons are relatively small polypeptide proteins which are secreted by most animal cells in response to exposure to a variety of inducers. Because of their antiviral, antiproliferative and immunomodulatory properties, interferons are of great interest as therapeutic agents. They exert their cellular activities by binding to specific membrane receptors on the cell surface. Once bound to the cell membrane, interferons initiate a complex sequence of intracellular events. In vitro studies demonstrated that these include the induction of certain enzymes, suppression of cell proliferation, immunomodulating activities such as enhancement of the phagocytic activity of macrophages and augmentation of the specific cytotoxicity of lymphocytes for target cells, and inhibition of virus replication in virus-infected cells. Thus, interferon proteins are functionally defined, and a wide variety of natural and synthetic or recombinant interferons are known. There are three major types of human IFNs. These are:

Leukocyte IFN or IFN-alpha, a Type 1 IFN produced in vivo by leukocytes.

Fibroblast IFN or IFN-beta, a Type 1 IFN produced in vivo by fibroblasts.

Immune IFN or IFN-gamma, a Type 2 IFN produced in vivo by the immune system.

IFN-beta is of particular interest for the treatment of a number of diseases or disorders, and especially in the treatment of multiple sclerosis or MS. Natural human IFN-beta is a 166 amino acid glycoprotein, and the encoding gene has been sequenced by Taniguchi, et. al., 1980, Gene 10: 11-15, and R. Derynck, et al., supra. Natural IFN-beta has three cysteine (cys) residues, located at amino acid positions 17, 31 and 141, respectively. In addition, numerous recombinant variants of IFN-beta are known.

Three recombinant IFN-beta products are licensed in Europe and the U.S. for treatment of MS. These are interferon beta-1a (“IFN-beta-1a”) or Avonex® (Biogen, Inc., Cambridge, Mass.), another IFN-beta-1a product marketed as Rebif® (Ares-Serono, Norwood, Mass.) and Ser₁₇ interferon-beta-1b (“IFN-beta-1b_{Ser17}”) or Betaseron® (Berlex, Richmond, Calif.).

IFN beta-1a is produced in mammalian cells, e.g., Chinese Hamster Ovary (“CHO”) cells using the natural human gene sequence, and the produced protein is glycosylated. See, for example, U.S. Pat. Nos. 5,795,779, 5,376,567 and 4,966,843, incorporated by reference herein. IFN beta-1b Ser₁₇ differs structurally from IFN-beta1a (Avonex® and Rebif®) because it is produced in Escherichia coli (“E. coli”) using a modified human gene sequence having an engineered cysteine-to-serine substitution at amino acid position 17, so that the protein is non-glycosylated. See, e.g. U.S. Pat. Nos. 4,588,585 and 4,737,462, the disclosures of which are incorporated by reference herein.

Both Rebif® and Avonex® are stated by their package inserts to have specific activities, by differing methods, of at least 2-3×10⁸ international units (IU)/mg. The Betaseron® package insert reports a specific activity of approximately 3×10⁷ IU/mg, indicating a ten-fold difference in potency. While these activities are determined by somewhat different methods, the order of magnitude differences in antiviral and antitumor activities are also reflected in the recommended doses, which are measured in micrograms (60-130 mcg/week) for the Rebif® and Avonex® glycosylated IFN-beta 1a products, and from 0.25 milligrams and up for the non-glycosylated Betaseron® IFN-beta 1b.

IFN-beta, in each of its recombinant formulations, has multiple effects on the immune system, including the ability to inhibit viral replication. IFN-beta-1b is described by the manufacturer (Berlex, Richmond, Calif.) as enhancing suppressor T cell activity, reducing proinflammatory cytokine production, down-regulation of antigen presentation, and inhibition of lymphocyte trafficking into the central nervous system. Other sources have reported that IFN-beta reduces the production of IFN-gamma by T-lymphocytes. Other beneficial therapeutic effects are also suspected.

However, as with all protein therapeutics, the drug is rapidly cleared from the bloodstream by nonspecific mechanisms, including renal filtration. In addition, patients injected with IFN-beta develop anti-IFN-beta neutralizing antibodies (“Nabs”). Nabs are a subset of binding antibodies that work to inhibit the normal biological effects of the eliciting antigen, and if elicited by a therapeutic protein, may reduce treatment efficacy. The risk of anti-IFN-beta Nab development and subsequent effects on treatment, may preclude early treatment of MS with interferon drugs—a consequence that would significantly curtail the therapeutic promise of these agents. Each of the marketed interferon beta drugs is associated with the development of Nabs in clinical trials during treatment of MS. In one two-year study of Betaseron®, nearly half of the treated patients, in both high- and low-dose groups, developed Nabs at some point in the study.

Some further disadvantages of the interferon therapeutics are physical instability i.e. protein aggregation, denaturation and precipitation to name a few and chemical instability, i.e. deamidation, hydrolysis, disulfide exchange, oxidation etc. Protein aggregation as used herein refers to the formation of dimers, trimers, tetramers, or multimers from monomers which may or may not precipitate in the formulation buffers and conditions of the present invention. The formulation of Ribif®, Avonex® and Betaseron® presently involve the use of HSA which can contribute to viral contamination as well as aggregation of the protein.

Polymer conjugates of IFN-beta's are known. U.S. Pat. Nos. 4,766,106 and 4,917,888, incorporated by reference herein, describe, inter alia, amide-linked IFN-beta 1b conjugates using mPEG-N-succinimidyl glutarate or mPEG-N-succinimidyl succinate. The patentees disclose that PEGylation of the protein is done using relatively high molar excesses of the activated polymer. Although linkage of the polymer to Lys residues is preferred, N-terminal-polymer linkages as well as those involving Cys, Glu and Asp are also disclosed. See column 8, lines 34-40 of the '888 patent, for example. Published PCT patent application No. WO99/55377 which describes site-selective modification of IFN-beta 1a at Cys-17 using a thiol-reactive PEGylating agent, describes shortcomings with the '106 and '888 results, however. Specifically, page 4, lines 5-18 of the published PCT application state that although non-specific PEGylation using large molar excesses of activated PEG provided conjugates improved solubility, “a major problem was the reduced level of activity and yield”.

Commonly assigned U.S. Pat. No. 5,738,846 discloses preparing various PEG-interferon conjugates. Column 14, line 1 thereof mentions IFN-beta as a suitable interferon for conjugation with various forms of activated PEG. Fractionation of the PEGylated product to recover specific species including the mono-PEGylated conjugates is also disclosed.

U.S. Pat. No. 5,109,120, incorporated by reference herein, describes methods of making PEG conjugates having an imidoester linker, including generally, IFN-beta. U.S. Pat. No. 6,531,122, describes IFN-beta variants or muteins different from IFN-beta 1b optionally conjugated to polymers such as PEG, including linkage via engineered Cys or Lys residues. Pepinsky, et al. in published U.S. Patent Appl. No. 20030021765 describe IFN-beta 1a polymer conjugates, including PEG conjugates and uses thereof. However, a 20 kDa N-terminal PEGylated IFN-beta 1a conjugate failed to provide prolonged effects on a biological marker for IFN-beta activity, despite prolonged presence in the serum of test animals (Pepinsky et al., 2001, The Journal of Pharm. and Exper. Ther. 297(3): 1059-1066). In addition, the Pepinsky report noted that a 30 kDa N-terminal PEGylated IFN-beta 1a conjugate retained only one-sixth of the activity of the 20 kDa N-terminal PEGylated IFN-beta 1a conjugate and a 40 kDa N-terminal PEGylated IFN-beta 1a conjugate lost all interferon activity.

Despite the foregoing, it should be noted that the various types of IFN proteins exhibit significant homology differences. For example, IFN-alpha and IFN-beta exhibit an average homology of only 3% in the domain of the signal sequence and of only 45% in the IFN polypeptide sequence, e.g., as described by Derynck, 1980 Nature, 285: 542-547. In addition, even though there is greater homology among the IFN-beta's, there are nonetheless some significant differences between the two, both in terms of therapeutic use, indications, etc.

In spite of the above-described disclosures, there remains a longstanding and heretofore unsolved need in the art for improved polymer-conjugated IFN-beta compositions, particularly those containing IFN-beta 1b. There also continues to be a need for improved compositions containing polymer-conjugated IFN-beta 1b wherein the polymer has a molecular weight of about 30 kDa (number average), or greater and which are free of human serum albumin (“HSA”).

### SUMMARY OF THE INVENTION

The above-described needs are addressed, and other advantages are provided, by the polymer-conjugated IFN-beta compositions described herein. In one aspect of the invention, there is provided an improved biologically-active polymer-interferon conjugate composition. The composition includes an interferon-beta 1b conjugated to a polyalkylene oxide (PAO) polymer having a molecular weight of at least about 12 kDa. Preferably, the PAO is a polyethylene glycol (PEG) having a molecular weight of from about 12kDa to about 60 kDa. More preferably, the PEG has a molecular weight of from about 30 kDa to about 60 kDa.

In one aspect of the invention, the polymer is linked to amino terminal of the IFN-beta 1b, while in other separate and preferred aspects of the invention the polymer is attached via an epsilon amino group of a Lys of the IFN-beta 1b. Depending upon the site of attachment and molecular weight of the polymer selected, retained anti-viral activities for the conjugates will range from at least about 65 percent for the 30 kDa polymer conjugates and at least about 15% for the 40 kDa polymer conjugates. In both cases, the amount of retained activity is significantly greater than that which was expected.

In certain optional embodiments, more than one polymer is linked to each IFN-beta 1b molecule. Preferably, the number of polymers linked to each IFN-beta 1b molecule ranges from one to about 4, and more preferably from 1 to about 3.

The composition of the present invention incorporates the polymer conjugate described above in the presence of certain buffers and excipients to increase the physical and chemical stability. A further improvement involves providing a formulation that is free of human serum albumen (“HSA”), in order to reduce the risk of viral contamination and protein aggregation.

In a still further improvement, the invention provides for an improved process for conjugating a protein with a non-antigenic polymer, such as a polyalkylene oxide in the presence of Zwittergent®. Previously, removal of Zwittergent® from such a reaction mixture has proved to be impractical. However, the present invention provides an economical method of separating the Zwittergent® from a polymer-conjugated protein, e.g., IFN-beta 1b, under acidic conditions.

Other aspects of the invention include methods of making the conjugate compositions or formulations as well as methods of treatment using the same.

As a result of the present invention there are provided improved IFN-beta 1b polymer conjugate compositions. The retained anti-viral activity of the conjugates of the present invention is surprising high, especially in view of the fact that the polymer portion thereof is in most aspects of the invention at least about 30 kDa. The prior art, see Pepinsky et al., 2001, The Journal of Pharm. and Exper. Ther. 297(3): 1059-1066, supra, which reported that EN-beta 1a, a glycosylated and more potent form of IFN-beta as compared to IFN-beta 1b, was substantially less active or inactive when the same molecular weight types of PEG were used.

In a related aspect of the invention improved methods of preparing a polyalkylene oxide-protein conjugate with a poorly soluble protein are provided, comprising the steps of

(a) solubilizing a protein of interest in a compatible aqueous solution in the presence of a protein-solubilizing amount of a compatible detergent;

(b) reacting the solubilized protein of interest with an activated polyalkylene oxide polymer, to produce a solution comprising a polyalkylene oxide-protein conjugate and the detergent;

(c) adjusting the reacted solution of step (b) to a pH effective to dissociate the detergent from the polyalkylene oxide-protein conjugate;

(d) separating the dissociated detergent from the polyalkylene oxide-protein conjugate, and recovering the polyalkylene oxide-protein conjugate.

The inventive method is optionally applied to any protein, and preferably to a protein that is poorly soluble in aqueous solution. More preferably, the protein is an interferon, such as interferon beta.

Preferably, the pH is adjusted in step (c) to a range from about pH 3 to about pH 4.

The activated polyalkylene oxide polymer is, for example, a polyethyelene glycol polymer ranging in size from about 12 kDa to about 60 kDa.

The detergent is optionally selected from an ionic detergent, a non-ionic detergent, a zwitterionic detergent, and combinations thereof. Preferably, the detergent is a zwitterionic detergent.

### BRIEF DESCRIPTION OF THE DRAWINGS

FIG. 1 is a graph showing comparative data discussed in Example 7.

The bars are represented as follows:
□ week 2, [symbol] week 3, [symbol] week 4, and ▪ week 6.

FIG. 2 is a graph illustrating the comparative data discussed in Example 8. The curves are represented as follows:

### IFN-beta-1b, PEG2-40k, PEG-UA-40k, and PEG-20k.

FIG. 3 is a graph showing the mean IFN-beta serum concentrations, by ELISA, in male and female Cynomolgus monkeys following injection of 15 μg/kg EZ-2046, as described by Example 9. The curves are represented by the following symbols, wherein “IM” is intramuscular, “IV” in intravenous, “SC” is subcutaneous, “F” is female and “M” is male.
[symbol] IM_F [symbol] IV-M
[symbol] IM_M [symbol] SC_F
[symbol] IV_F [symbol] SC_M

FIG. 4 illustrates the purity of the final product of PEG-IFN-beta 1b, as determined by RP-HPLC using an ELSD detector, as described by Examples 2 and 3.

### DETAILED DESCRIPTION OF THE INVENTION

Accordingly, the invention provides a composition comprising:

a) an interferon conjugated to a polyalkylene oxide polymer having a molecular weight of at least about 12 kDa, or alternatively, at least about 20 kDa; and optionally

b) a surfactant;

c) an excipient, and

d) a buffer, wherein the pH range of the solution is from about 3.0 to about 11.

In the embodiments herein the compositions have a pH from about 3.0 to about 8.0.

In other embodiments, the inventive compositions have a pH from about 3.0 to about 5.0, with a pH of from about 3.0 to about 4.0 being most preferred. The ionic strength of the compositions provided herein has been found to affect the stability i.e. prevent aggregation. Low ionic strength is preferred in low pH buffers while high ionic strength is preferred in high pH buffers. In one embodiment, the ionic strength of a composition on the invention having a pH of from about 3.0 to about 4.0 is lower than 10 mM. In another embodiment, the ionic strength of a composition of the invention having a pH of from about 5.5 to about 7.5 is about 100 to about 150 mM.

The interferon used in the compositions of the invention is preferably interferon-beta 1b and more preferably IFN-beta-1b_{Ser17}.

### A. Beta Interferons

The term “interferon-beta” or “IFN-beta” as used herein refers to IFN-beta isolated from natural sources and/or produced by recombinant DNA technology as described in the art, having sequence homology with, and the functionality, including bioactivity, of native IFN-beta. The term “interferon-beta 1b” or “IFN-beta 1b” as used herein refers to a mutein of IFN-beta having residue Cys₁₇ replaced by residue Ser₁₇, and expressed in a nonglycosylated form, with the N-terminal amino acid, Methionine, post-translationally removed, and represented herein as SEQ ID NO:1.

As noted in more detail, supra, the beta interferon (IFN-beta) portion of the polymer conjugate can be prepared or obtained from a variety of sources, including recombinant techniques such as those using synthetic genes expressed in suitable eukaryotic or prokaryotic host cells, e.g., see U.S. Pat. No. 5,814,485, incorporated by reference herein. In addition, the IFN-beta can also be a mammalian source extract such as human, ruminant or bovine IFN-beta. One particularly preferred IFN-beta is IFN-beta-1b_{Ser17}, a recombinantly-made product available from Berlex, (Richmond, Calif.), as described by U.S. Pat. No. 4,737,462, incorporated by reference.

The IFN-beta proteins employed to produce the inventive conjugates were either obtained commercially, e.g., IFN-beta 1b was obtained from Berlex, Inc. (Richmond, Calif.) or produced and isolated as exemplified hereinbelow. Native human IFN-beta is optionally employed, although it is preferred to use an IFN-beta mutein optimized for production and solubilization in a prokaryotic host. One preferred prokaryotic host cell is Escherichia coli.

Many muteins of the native human or animal IFN-beta are known and contemplated to be employed in the practice of the invention. Preferred muteins are described in greater detail by U.S. Pat. Nos. 4,588,585, 4,959,314, 4,737,462 and 4,450,103, incorporated by reference herein. In brief, as noted above, a preferred mutein is one wherein the Cys₁₇ residue of native human IFN-beta is replaced by serine, threonine, glycine, alanine, valine, leucine, isoleucine, histidine, tyrosine, phenylalanine, tryptophan or methionine. Most preferred is the non-glycosylated Ser₁₇ mutein of IFN-beta, also referred to herein as IFN-beta 1b.

Numerous methods of expressing and isolating IFN-beta proteins from prokaryotic host systems, and vectors suitable for expression by prokaryotic host cells, are known. For example, much of the IFN-beta employed in the examples provided hereinbelow was produced by the following method. A synthetic gene encoding an IFN-beta, e.g., IFN-beta 1b, was synthesized, following codon optimization for bacterial expression.

Other methods and reagents for IFN-beta production and purification are described, for example, by U.S. Pat. Nos. 6,107,057, 5,866,362, 5,814,485, 5,523,215, 5,248,769, 4,961,969, 4,894,334, 4,894,330, 4,748,234, 4,656,132, all incorporated by reference herein, as well as by other references too numerous to mention.

Methods of expressing and isolating IFN-beta proteins, and vectors suitable for expression by eukaryotic host cells, such as Chinese Hamster Ovary (“CHO”) cells, are described in detail, e.g., by U.S. Pat. Nos. 4,966,843, 5,376,567, and 5,795,779, incorporated by reference herein.

### B. Non-Antigenic Polymers

The polymeric portion of the conjugate useful in the compositions of the invention can be linear and is preferably selected from the group consisting of:

A-O—(CH₂CH₂O)ₓ—

A-O—(CH₂CH₂O)ₓ—CH₂C(O)—O—,

A-O—(CH₂CH₂O)ₓ—CH₂CH₂NR₇—,

A-O—(CH₂CH₂O)ₓ—CH₂CH₂SH,

—O—C(O)CH₂—O—(CH₂CH₂O)ₓ—CH₂C(O)—O—,

—NR₇CH₂CH₂—O—(CH₂CH₂O)ₓ—CH₂CH₂NR₇—,

—SHCH₂CH₂—O—(CH₂CH₂O)ₓ—CH₂CH₂SH—,

wherein

A is a capping group;

R₇ is selected from hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₄ alkenyls, C₃₋₁₂ branched alkenyls, C₁₋₆ alkynyls, C₃₋₁₂ branched alkynyls, C₁₋₆ heteroalkyls, substituted C₁₋₆ hetero-alkyls, C₁₋₆ alkoxyalkyl, phenoxyalkyl and C₁₋₆heteroalkoxys, and

x is the degree of polymerization. The variable x is preferably a positive integer selected so that the molecular weight of the polymer is within the ranges disclosed herein, i.e. from about 20 to about 60 kDa, as being preferred.

Alternatively the polymeric portion of the conjugate useful in the compositions of the invention can be branched and is preferably selected from the group consisting of:

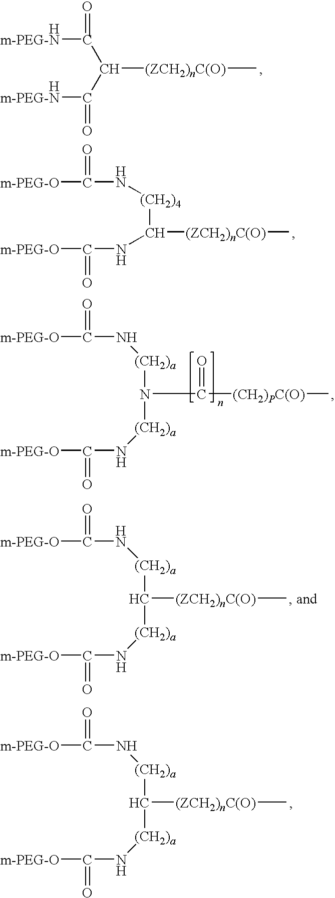

wherein:
(a) is an integer of from about 1 to about 5;
Z is O, NR₈, S, SO or SO₂; where R₈ is H, C₁₋₈ allyl, C₁₋₈ branched alkyl, C₁₋₈ substituted alkyl, aryl or aralkyl;
(n) is 0 or 1;
(p) is a positive integer, preferably from about 1 to about 6, and m-PEG is CH3-O—(CH₂CH₂O)ₓ—.

Preferably, the capping group A is selected from the group consisting of OH, CO₂H, NH₂, SH, and C₁₋₆ alkyl moieties.

More preferably, interferon beta 1b is conjugated to a polyalkylene oxide polymer selected from the group selected from:

A-O—(CH₂CH₂O)_{X}—

-A-O—(CH₂CH₂O)ₓ—CH₂C(O)—O—,

A-O—(CH₂CH₂O)ₓ—CH₂CH₂NR₇—,

A-O—(CH₂CH₂O)ₓ—CH₂CH₂SH,

—O—C(O)CH₂—O—(CH₂CH₂O)ₓ—CH₂C(O)—O—,

—NR₇CH₂CH₂—O—(CH₂CH₂O)ₓ—CH₂CH₂NR₇—,

—SHCH₂CH₂—O—(CH₂CH₂O)ₓ—CH₂CH₂SH—,

wherein

A is a capping group;

R₇ is selected from hydrogen, C₁₋₆alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆alkenyls, C₃₋₁₂ branched alkenyls, C₁₋₆alkynyls, C₃₋₁₂ branched alkynyls, C₁₋₆heteroalkyls, substituted C₁₋₆hetero-alkyls; C₁₋₆alkoxyalkyl, phenoxyalkyl and C₁₋₆heteroalkoxys, and

x is the degree of polymerization. The variable x is preferably a positive integer selected so that the molecular weight of the polymer is within the ranges disclosed herein, i.e. 20-60 kDa, as being preferred.

Alternatively the polymeric portion of the conjugate useful in the compositions of the invention can be branched and is preferably selected from the group consisting of:

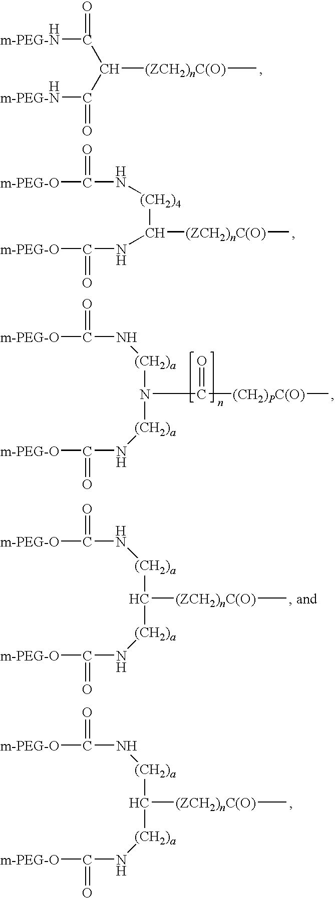

wherein:
(a) is an integer of from about 1 to about 5;
Z is O, NR₈, S, SO or SO₂; where R₈ is H, C₁₋₈ alkyl, C₁₋₈ branched alkyl, C₁₋₈ substituted alkyl, aryl or aralkyl;
(n) is 0 or 1;
(p) is a positive integer, preferably from about 1 to about 6, and m-PEG is CH₃—O—(CH₂CH₂O)_{X}—.

Preferably, the capping group A is selected from the group consisting of OH, CO₂H, NH₂, SH, and C₁₋₆ alkyl moieties.

More preferably, interferon beta 1b is conjugated to a polyalkylene oxide polymer selected from the group selected from:

A-O—(CH₂CH₂O)ₓ—

A-O—(CH₂CH₂O)ₓ—CH₂C(O)—O—,

A-O—(CH₂CH₂O)ₓ—CH₂CH₂NR₇—,

A-O—(CH₂CH₂O)ₓ—CH₂CHₓSH,

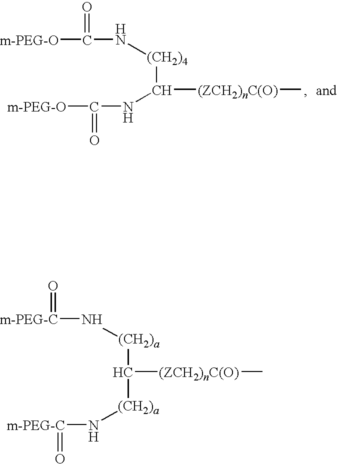

wherein the molecular weight of the polyalkylene oxide polymer ranges from about 20-40 and preferably 30 kDa to about 40 kDa.

In order to conjugate the IFN-beta to polymers such as poly(alkylene oxides), one of the polymer hydroxyl end-groups is converted into a reactive functional group which allows conjugation. This process is frequently referred to as “activation” and the product is called an “activated” polymer or activated poly(alkylene oxide). Other substantially non-antigenic polymers are similarly “activated” or functionalized. Polyethylene glycol (PEG) is the most preferred PAO. The general formula for PEG and its derivatives, i.e.

A′-O—(CH₂CH₂O)ₓ-A

where (x) represents the degree of polymerization or number of repeating units (up to about 2300) in the polymer chain and is dependent on the molecular weight of the polymer. (A) is an activated linking group such as those described below while A′ is the same as (A), an alternative activated linking group, H or a capping group such as CH₃. Such mono-activated PEG derivatives are commonly referred to as MPEG derivatives. In addition to mPEG, it should be generally understood that PEGs terminated on one end with any C₁₋₄ alkyl group are also useful.

In alternative aspects, the polymer is a poly(propylene glycol) or PPG. Branched PEG derivatives such as those described in commonly-assigned U.S. Pat. Nos. 5,643,575, 5,919,455 and 6,113,906, “star-PEG's”, terminally-branched or forked PEG's and multi-armed PEG's such as those described in Nektar catalog “Polyethylene Glycol and Derivatives for Advanced PEGylation 2003”. The disclosure of each of the foregoing is incorporated herein by reference. A non-limiting list of PEG derivatives is provided includes: mPEG-A, A-PEG-A, and

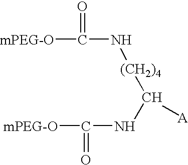

A non-limiting list of suitable PEG activated linking groups is provided below. The activated linking groups correspond to A in the formula given above.

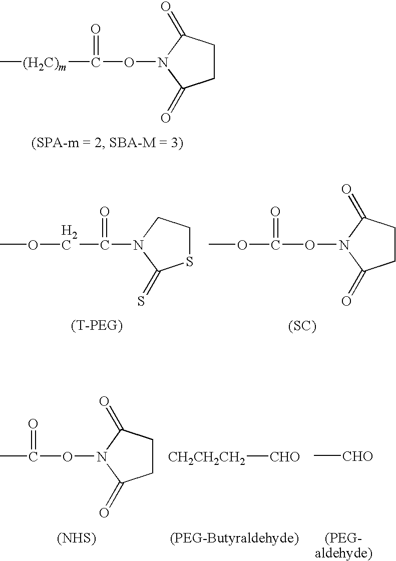

The foregoing can be attached to an alpha and/or omega terminal of the PEG, it being understood that when both such linking groups are employed, the resulting conjugates can have two (2) equivalents of IFN-beta per unit of polymer.

As will be appreciated by those of ordinary skill, the aldehyde derivatives are used for N-terminal attachment of the polymer to the IFN. For example, polyalkylene oxide (PAO) aldehydes react only with amines and undergo reductive amination reactions with primary amines in the presence of sodium cyanoborohydride to form a secondary amine. Suitable polyethylene glycol (PEG) aldehydes are available from Nektar of San Carlos, Calif. In other aspects of the invention, the other activated linkers shown above will allow for non-specific linkage of the polymer to Lys amino groups-forming carbamate (urethane) or amide linkages.

In some preferred aspects of the invention when Lys attachment is desired, the activated linker is an oxycarbonyl-oxy-N-dicarboximide group such as a succinimidyl carbonate group; Alternative activating groups include N-succinimide, N-phthalimide, N-glutarimide, N-tetrahydrophthalimide and N-norborene-2,3-dicarboxide. These urethane-forming groups are described in commonly owned U.S. Pat. No. 5,122,614, the disclosure of which is hereby incorporated by reference. Other urethane-forming activated polymers such as benzotriazole carbonate activated (BTG-activated PEG—available from Nektar) can also be used. See also commonly-assigned U.S. Pat. No. 5,349,001 with regard to the above-mentioned T-PEG.

It will also be appreciated that heterobifunctional polyalkylene oxides are also contemplated for purposes of cross-linking IFN-beta, or providing a means for attaching other moieties such as targeting agents for conveniently detecting or localizing the polymer-IFN-beta conjugate in a particular areas for assays, research or diagnostic purposes.

In many aspects, suitable polymers will vary somewhat by weight but are preferably at least about 20,000 (number average molecular weight). Alternatively, the polymers may range from about 20,000 to about 60,000, with from about 30,000 to about 40,000 being preferred. In some aspects of the invention where bifunctional PEG is used, the molecular weight can be as low as 20,000.

As an alternative to the preferred PAO-based polymers, other effectively non-antigenic, terminally functionalized polymers such as dextran, polyvinyl alcohols polyvinyl pyrrolidones, polyacrylamides such as HPMA's-hydroxypropylmethacryl-amides, polyvinyl alcohols, carbohydrate-based polymers, copolymers of the foregoing, and the like can be used if the same type of activation is employed as described herein for PAO's such as PEG. Those of ordinary skill in the art will realize that the foregoing list is merely illustrative and that all polymeric materials having the qualities described herein are contemplated. For purposes of the present invention, “effectively non-antigenic” and “substantially non-antigenic” shall be understood to include all polymeric materials understood in the art as being substantially non-toxic and not eliciting an appreciable immune response in mammals.

The activated polymers are reacted with IFN-beta under conditions suitable to permit attachment at protein sites that do not significantly interfere with biological activity, e.g., so that the conjugated IFN-beta retains antiviral and other desirable biological activity. Histidine groups, free carboxylic acid groups, suitably activated carbonyl groups, oxidized carbohydrate moieties and mercapto groups, if available on the IFN-beta of interest, can also be used as supplemental attachment sites, when appropriate.

In one embodiment, the PEG-IFN-beta-1b conjugate of the composition is present at a concentration of from about 0.01 mg/ml to about 4.0 mg/ml. In other embodiments the protein conjugate is present at a concentration of from about 0.05 mg/ml to about 3.0 mg/ml.

### C. Solubilization of Proteins for Conjugation Reaction by Detergent

In order for a polyalkylene oxide polymer to undergo a useful conjugation reaction with a protein of interest, the protein must be in solution. Unfortunately, many of the proteins that are desirable to react with polyalkylene oxide polymers are difficult to maintain in aqueous solution under conditions that are compatible with conjugation reaction conditions. This is a problem with many insoluble proteins, including IFN-beta-1b.

One non-destructive method for solubilizing proteins is to include a surfactant or detergent in the solution. A detergent will solubilize an otherwise insoluble protein in aqueous solution by associating with the protein and preventing precipitation and/or aggregation. A number of ionic, nonionic and zwitterionic detergents are well suited for protein solubilization, but before the present invention, effectively separating the associated detergent from the reaction product, i.e., from the produced polymer conjugated protein, has been a significant obstacle.

The present invention provides a new and efficient method for separating a detergent from conjugated proteins. In brief, the protein of interest is solubilized with a compatible detergent and subjected to a conjugation reaction. After the conjugation reaction is complete, the pH of the reaction solution is lowered sufficiently to dissociate the detergent from the protein. The lowered pH ranges, e.g., from about pH 3 to about pH 4. Thereafter, the detergent is physically separated from the conjugated protein, e.g., by centrifugation and/or filtration.

Preferably, the separation step is by diafiltration, so that the conjugated protein is retained by the diafilter, while the detergent is removed by washing in an excess of a compatible buffer. The diafilter is preferably of a 10K size, although the artisan will appreciate that this can be varied with the size of the polymer-conjugated protein of interest.

Preferred detergents for stabilizing or solubilizing a relatively insoluble protein in aqueous solution include ionic, nonionic and zwitterionic detergents. Ionic detergents are those containing a head group with a net charge. These either contain a hydrocarbon (alkyl) straight chain, as in sodium dodecyl sulfate (SDS), or a rigid steroidal structure as in the deoxycholate-based detergents, e.g., sodium deoxycholate.

Non-ionic detergents contain uncharged, hydrophilic head groups that consist of either polyoxyethylene moieties. Exemplary non-iionic detergents include polyoxyethylene derivatives, such as polyoxyethylene lauryl ether (e.g., BRIJ®), Glucamide-based detergents, such as octyl dodecanol (TRITONX-100®, and ethoxylated fatty acid esters (e.g., TWEENs®) or glycosidic groups such as in octyl glucoside and dodecyl maltoside.

Zwitterionic detergents do not contain a net charge. Zwitterionic detergents include those provided by Anatrace as Anzergent® or by Calbiochem ZWIMTERGENT®. Preferred zwitterionic detergents are the ZWITTERGENT® 3-X-series and CHAPS. More preferred is ZWITTERGENT® 3-14, as described by Examples 2 and 3, hereinbelow. Several additional particular detergents contemplated to be employed in the above-described process are listed in the following table.

| | Type of |
|---|---|
| Exemplary Detergents* | Detergent |
| Cetyltrimethylammonium Bromide (CTAB) | Cationic |
| CHAPS | Zwitterionic |
| Cholic Acid, Sodium Salt | Anionic |
| n-Dodecyl-beta-D-maltoside | Non-ionic |
| n-Hexyl-beta-D-glucopyranoside | Non-ionic |
| Lauryldimethylamine Oxide | Zwitterionic |
| n-Octyl-beta-D-glucopyranoside | Non-ionic |
| Sodium Dodecyl Sulfate (SDS) | Anionic |
| n-Tetradecyl-beta-D-maltoside | Non-ionic |
| TRITON ® X-100 Detergent (Av.) | Non-ionic |
| TWEEN ® 20, (Av.) | Non-ionic |
| PROTEIN GRADE ® Detergent | |

*Reference: A Guide to the Properties and Uses of Detergents in Biology and Biochemistry by Calbiochem, incorporated by reference herein in its entirety.

The optimal concentration of detergent or surfactant will vary with the protein of interest and the particular detergent or surfactant that is selection, but is preferably determined to be the lowest concentration needed to keep the protein of interest safely in aqueous solution.

It is contemplated that this detergent removal process has utility for supporting the polymer conjugation of a range of useful proteins of otherwise limited aqueous solubility. Such proteins generally include lipoproteins or membrane-bound proteins such as interleukin-2 (IL-2). Preferably, the protein is an IFN, such as IFN beta 1b.

### D. Buffers, Surfactants and Excipients

The compositions of the present invention contain a buffer which may be selected from the group consisting of Glycine-HCl, acetic acid, sodium acetate, sodium aspartate, sodium citrate, sodium phosphate and sodium succinate.

Preferably, the buffer is selected from sodium acetate, sodium citrate and glycine HCl. In addition, the buffer preferably has an ionic strength of about 10 mM and is present in a concentration of from about 1 mM to about 10 mM. Preferably the buffer is present at a concentration of from about 3 mM to about 5 mM.

The compositions of the present invention also contain an excipient wherein the excipient is non-ionic and is selected from the group consisting of, monosaccharides, disaccharides, and alditols.

Preferably, the excipient is selected from the group consisting of monosaccharides such as, glucose, ribose, galactose, D-mannose, sorbose, fructose, xylulose, and the like, disaccharides such as, sucrose, maltose, lactose, trehalose and the like, polysaccharides such as, raffinose, maltodextrins, dextrans, and the like and alditols such as glycerol, sorbitol, mannitol, xylitol, and the like.

More preferably, the excipient is selected from the group consisting of sucrose, trehalose, mannitol and glycerol or a combination thereof with the group consisting of mannitol and sucrose or a combination thereof being most preferred.

For the compositions of the present invention, mannitol can be present at a concentration of from between 1% to about 6%, sucrose can be present in a concentration from about 8% to about 10% and trehalose can be present in a concentration of from about 8% to about 10%. Preferably, the compositions contain about 5% mannitol or about 9% sucrose or 9% trehalose.

The compositions of the present invention further contain a surfactant, wherein the surfactant is non-ionic and is selected from the group consisting of polysorbate 80 (Tween 80), polysorbate 20 (Tween 20), and polyethylene glycol. In one embodiment, the surfactant is polysorbate 80. In one embodiment, the surfactant Tween 80 is present at a concentration of from about 0.01% to about 0.5%. Preferably, for compositions of the present invention, Tween 80 is present in a concentration of about 0.5%.

### Reaction Conditions

Details concerning specific reaction conditions which are suitable for making monoPEGylated compounds are provided in the examples. However, the processes of the present invention generally include reacting interferon-beta 1b with an activated polyalkylene oxide polymer having a molecular weight of at least about 30 kDa under conditions sufficient to cause conjugation of the activated polyalkylene oxide polymer to the interferon-beta 1b, and retaining at least a portion of the antiviral activity relative to native interferon-beta 1b, using the standard assay measurements. A non-denaturing surfactant, such as a non-ionic detergent or a zwitterionic detergent, was present as a component in the PEGylation reaction. The preferred surfactant is a zwitterionic detergent. The more preferred is a sulfobetaine, such as Zwittergent® 3-14. The reaction conditions for effecting conjugation further include conducting the attachment reaction with from about equi-molar to about a relatively small molar excess of the activated polymer with respect to the IFN. In this regard, the process can be carried out with about 1-15-fold molar excess; preferably about 2-12-fold molar excess and most preferably about 3-10-fold molar excess. The conjugation reaction can be carried out at about room temperature, 20-25° C. It is also preferred that the coupling reaction be allowed to proceed for rather short periods of time, i.e. 0.5-2 hours, before quenching. It was determined that reaction with the aldehyde-activated polymers was best conducted at pH of about 5.2, with later addition of the reducing agent, sodium cyanoborohydride. In practice, the non-aldehyde-activated polymers result in the formation of a mixture of polymer-IFN positional isomers. Preferably, each isomer contains a single polymer strand attached to the interferon via an amino acid residue. In alternative embodiments, there can be more than one strand of polymer attached to the IFN as a result of the Lys directed processes. Solutions containing these conjugates are also useful as is or can be further processed to separate the conjugates on the basis of molecular weight.

Due to the nature of the solution-based conjugation reactions, the Lys-attached compositions are a heterogeneous mixture of species which contain the polymer strand(s) attached at different sites on the interferon molecule. In any solution containing the conjugates, it is likely that a mixture of at least about 2, preferably about 6 and more preferably about 8 positional isomers will be present.

### Methods of Treatment

Another aspect of the present invention provides methods of treatment for various medical conditions in mammals, preferably humans. The methods include administering an effective amount of a pharmaceutical composition that includes an IFN-beta-polymer conjugate prepared as described herein, to a mammal in need of such treatment. The conjugates are useful for, among other things, treating interferon-susceptible conditions or conditions which would respond positively or favorably as these terms are known in the medical arts to interferon-based therapy.

Conditions that can be treated in accordance with the present invention are generally those that are susceptible to treatment with IFN-beta. For example, susceptible conditions include those which would respond positively or favorably as these terms are known in the medical arts to IFN-beta-based therapy. Exemplary conditions which can be treated with IFN-beta include, but are not limited to, multiple sclerosis and other autoimmune disorders, cell proliferation disorders, cancer, viral infections and all other medical conditions know to those of ordinary skill to benefit from interferon-beta and/or interferon-beta 1b therapy. In a preferred aspect of the invention, the polymer conjugated IFN-beta is administered to patients in amounts effective to treat multiple sclerosis.

A further aspect of the invention provides for the treatment of conditions that can be treated with polymer-conjugated IFN-beta, and preferably polymer-conjugated IFN-beta 1b, that have heretofore not fully responded to such treatment because the negative side effects previously outweighed the benefits of the treatment at a given dosage. For example, IFN-beta has been tested for treating poor-prognosis Kaposi sarcoma related to HIV/AIDs infection Miles et al., 1990 Ann Intern Med. 112(8):582-9 and the data suggested a minimal potential benefit. Practice of the invention would allow treatment of this condition, and others, at higher doses and in combination with other art-known therapeutic agents.

### Methods of Administration

Administration of the described dosages may be every other day, but is preferably once or twice a week. Doses are usually administered over at least a 24 week period by injection or infusion. Administration of the dose can be intravenous, subcutaneous, intramuscular, or any other acceptable systemic method, including subdermal or transdermal injection via conventional medical syringe and/or via a pressure system. Based on the judgment of the attending clinician, the amount of drug administered and the treatment regimen used will, of course, be dependent on the age, sex and medical history of the patient being treated, the stage or severity of the specific disease condition and the tolerance of the patient to the treatment as evidenced by local toxicity and by systemic side-effects. Dosage amount and frequency may be determined during initial screenings of neutrophil count.

The amount of the IFN-beta-polymer conjugate composition administered to treat the conditions described above is based on the IFN activity of the polymeric conjugate. It is an amount that is sufficient to significantly affect a positive clinical response. Although the clinical dose will cause some level of side effects in some patients, the maximal dose for mammals including humans is the highest dose that does not cause unmanageable clinically-important side effects. For purposes of the present invention, such clinically important side effects are those which would require cessation of therapy due to severe flu-like symptoms, central nervous system depression, severe gastrointestinal disorders, alopecia, severe pruritus or rash. Substantial white and/or red blood cell and/or liver enzyme abnormalities or anemia-like conditions are also dose limiting.

Naturally, the dosages of the various IFN-beta conjugate compositions will vary somewhat depending upon the IFN-beta moiety and polymer selected. In general, however, the conjugate is administered in amounts ranging from about 100,000 to about 1 to 50 million IU/m² per day, based on the condition of the treated mammal or human patient. The range set forth above is illustrative and those skilled in the art will determine the optimal dosing of the conjugate selected based on clinical experience and the treatment indication.

### EXAMPLES

The following examples serve to provide further appreciation of the invention but are not meant in any way to restrict the effective scope of the invention.

### Example 1

### Production of Recombinant IFN-beta 1b

### A. Optimized Gene Encoding IFN-beta 1b

A cDNA gene (SEQ ID NO: 2) encoding the reported 165 amino acid sequence of human interferon-beta-1b (SEQ ID NO: 1) was synthesized. This gene has codons optimized for expression in E. coli, and was synthesized using standard chemical synthesis of overlapping oligonucleotide segments. The flanking restriction sites, NdeI and BamHI, were included at the termini of the gene. Following digestion of the synthetic DNA with the restriction enzymes NdeI and BamHI, the 0.5 kilobase gene was ligated via T4 DNA ligase into the plasmid vector pET-27b(+) (Novagen Corporation), which had also been digested with these two enzymes. The recombinant plasmid was introduced into E. coli strain BLR (DE3) by electroporation using a BTX Electro Cell Manipulator 600 according to the manufacturer's instructions. The transformation mixture was plated on LB agar plates containing kanamycin (15 μg/ml) to allow for selection of colonies containing the plasmid pET-27b(+)/IFN-beta-1b (designated plasmid pEN831 in strain EN834). Isolated colonies were further purified by plating and analyzed for IPTG inducible gene expression by standard methods such as those described in Novagen pET System Manual Ninth Edition.

### B. Expression of IFN-beta 1b

The above described E. coli codon optimized gene for IFN-beta-1b was expressed in the BLR/pET system which employs the T7 RNA polymerase expression control. The IFN-beta-1b protein was expressed in inclusion bodies comprising about 30% of total cell protein. After solubilization and butanol extraction, the protein was purified to near homogeneity by DEAE and SP (Amersham) ion exchange chromatography in the presence of Zwittergent® 3-14. All other standard recovery steps were employed. Expression of betaseron was achieved by inducing the growing culture in the presence of IPTG, 1.0 mM, for 2-3 hours at 37° C. IFN-beta-1b was accumulated in the inclusion bodies.

C. Purification of Interferon-beta-1b from Inclusion Bodies

Purification of IFN-beta-1b from inclusion bodies was achieved to near homogeneity following modifications and amalgamations of previously published protocols. Briefly, IFN-beta-1b from inclusion bodies was solubilized in SDS, extracted into butanol phase and subsequently acid precipitated. Butanol extraction offered two-fold advantages in achieving high fold-purification in one step and by removing majority of the free SDS from the preparation. The acid precipitated protein was then resuspended in Zwittergent® and solubilized by transient pH shock from pH 12.0 to pH 8.0, carefully avoiding the amino-terminal deamidation process. IFN-beta-1b was then subjected to a critical renaturation step and two ion-exchange chromatographies to achieve maximum purity.

Alternatively, the IFN-beta-1b protein can be commercially obtained from Berlex Laboratories.

### Examples 2-3

### Preparation of PEG2-40k-IFN and PEG-UA-40k-IFN

In these Examples, activated PEG2-40k-IFN and PEG2-40k-beta alanine-NHS obtained from Nektar Therapeutics, Huntsville, Ala., and Enzon Pharmaceuticals, Inc., respectively, were each separately incubated with the IFN-beta of Example 1. With fast stirring, each amine activated PEG powder was separately added to 0.3-0.8 mg/mL IFN-beta (>95% purity) in ˜100-mL of 50-100 mM sodium phosphate, pH 7.8, 2 mM EDTA, and 0.05% Zwittergent® at 0.5-1.0 g/min. Alternatively, PEG powder was pre-dissolved in one tenth volume of IFN-beta solution in 1 mM HCl and add PEG solution to IFN-beta solution. The reaction molar ratio of PEG:IFN was 5-10:1. After 60-min reaction at 25° C., each reaction was quenched by lowering pH to 6.5 with 2 N HAc. The conjugation yield of mono PEG-IFN was 40-60%, as analyzed by RP-HPLC.

The reaction mixture was diluted with 0.03% Zwittergent® in H₂O to a conductivity of 5.8 mS/cm. A cation exchange resin, such as SP FF resin (Amersham Biosciences, NJ), was packed on a Waters AP-2 column to a bed height of 6 cm, ID 2 cm, CV 18.85 ml and equilibrated with 10 mM sodium phosphate, pH 6.5, 20 mM NaCl, 0.05% Zwittergent® 3-14. A sample of reaction mixture was loaded on the column at 50 cm/hr (˜4 mg protein was loaded per mL resin), washed with 1-1.5 column volume (CV) of column equilibration buffer until baseline and then with 5 CV of 10 mM sodium phosphate, pH 6.5, 60 mM NaCl to remove high MW conjugates.

The product was eluted out with 10 mM sodium phosphate, pH 6.5, 200 mM NaCl. Zwittergent® in eluent was removed by diafiltration using Millipore Labscale TFF system (two cartridges of regenerated cellulose 10K membranes “Pellicon XL cartridges” PLCTK 10 50 cm2, cat # PXC030C50 Lot# C3SN75289-023, LFL Tygon tubing with 6 mm (¼″) OD, 3 mm (⅛″) ID (Masterflex 06429-16, mfg by Saint-Gobain). The system settings were □P=4 psi, pump feed set at “1” with string speed at “2” to measure=18 psi, retentate=14 psi. The product that was eluted from an HS (Applied Biosystems) or SP (Amersham) column was immediately diluted with 10-fold of diafiltration buffer (5 mM HAc, pH 3.7) and then concentrated by 10-fold on the diafiltration system. The process consumed 50-fold of sample volume of diafiltration buffer for a complete removal of Zwittergent®. The formulation was conducted thereafter on the same system. Purity was confirmed by RP-HPLC chromatography. The parameters for the RP-HPLC analysis were as follows.
Column: Jupiter C5, 5 μm, 300 Å, 4.6×150 mm (Phenomenex, Calif.)
Column Temperature: 45° C.
Auto-sampler Thermostat: 4° C.
Mobile Phase A: 0.1% Trifluoroacetic Acid (TFA) and 10% 1,4-Dioxane in water
Mobile Phase B: 0.1% Trifluoroacetic Acid (IFA) and 10% 1,4-Dioxane in methanol;
Flow Rate: 1.0 mL/min

The RP-HPLC results are shown in FIG. 4, confining that the purity of the product was >95% pure mono PEG-IFN-beta 1b.

### Example 4

### di PEG-20k-IFN

The same PEGylation conditions employed in Examples 2 and 3 were employed as above, except the reaction molar ratio was about 1:20. After 60 min reaction with PEG-20k-SPA, obtained from Nektar Therapeutics, di PEG-20k-IFN was purified by a size exclusion column, followed by a cation exchange column.

### Example 5

### Methods to Detect Aggregation

The samples were buffer-exchanged to the buffers described in following table, using Centricon YM-30 (Millipore Corp., Bedford, Mass.). To accelerate the study, the samples were placed at 37° C. and under N₂ for 24 hrs. The stability was monitored on SEC-HPLC. Aggregation of sample particles was determined by size exclusion chromatography HPLC (Superdex 200, HR, Amersham Biosciences, Piscataway, N.J.), using a 0.1 M sodium phosphate, pH 6.8 buffer system,

RP-HPLC was employed to detect degradation. Non reducing SDS-PAGE and antiviral and antiproliferation activities were also employed

Aggregation is defined herein as a physical linking of one or more protein monomers to form dimer, trimer, tetramer, or multimers, that may or may not precipitate out of solution in the formulation buffers and the conditions that were examined. The soluble aggregate is converted to monomer on non reducing SDS gel, and will be reversed to monomer upon dilution.

### Liquid Formulation

### Example 5A

### The Lower pH of Formulation Buffer is Preferred

Organic and inorganic buffers, with a pH ranging from 3.0 to 11.0 were tested. Glycine-HCl, pH 3.0, acetic acid, pH 3.7, sodium acetate, pH 4.5, sodium succinate, pH 4.4, sodium aspartate, pH 5.4, sodium citrate, pH 3-6, and sodium phosphate, pH 6.0-7.4 were used as basic buffers for examining effects of excipients. In the presence of 3 mM HAc, pH 3.7, the conjugate was stable at 37° C. for at least 17 days.

Effect of Buffer pH on Aggregation*

| | | | | Protein | T | Time | Aggrega- |
|---|---|---|---|---|---|---|---|
| Buffer | Conc.** | Excipient | pH | (mg/ml) | (° C.) | (day) | tion (%) |
| Acetic | 3 mM | | 3.7 | 0.1 | 4 | 37 | 0 |
| acid | | | | | | | |
| Citrate | 5 mM | | 4.0 | | | | 0 |
| Citrate | 5 mM | | 5.0 | | | | 3.5 |
| Citrate | 5 mM | | 6.0 | | | | 4.1 |
| Na | 5 mM | | 7.4 | | | | 6.7 |
| Phospahte | | | | | | | |
| Na | 5 mM | | 8.5 | | | | 57.3 |
| phosphate | | | | | | | |
| H₂O | | | | | | | 4.7 |
| Acetic | 3 mM | mannitol, | 3.7 | 0.25 | 37 | 11 | 1.8 |
| acid | | 5% | | | | 26 | 6.7 |

*The percent aggregation was analyzed by SEC-HPLC.
**Concentration

As summarized by the above table, the preferred buffers are acetate (free acid or salt), citrate (free acid or salt), and glycine-HCl. Citrates have a dual role as chelating agents. The preferred pH is acidic, more specifically between pH 3.0 and 4.0. The preferred concentrations of the buffers at pH 3.0-4.0 are below 10 mM. Citrate buffers at >50 mM will result in excess pain on subcutaneous injection and toxic effects due to the chelation of calcium in the blood.

### Example 5B

### Excipients of Carbohydrates

Non-ionic tonicity modifying agents were examined as bulking agents to stabilize the conjugate and to render the compositions isotonic with body fluid. As classified in textbooks, monosaccharides include glucose, ribose, galactose, D-mannose, sorbose, fructose, xylulose, and the like; disaccharides are sucrose, maltose, lactose, trehalose, and the like, and polysaccharides comprise raffinose, maltodextrins, dextrans, and the like. Alditols contain glycerol, sorbitol, mannitol, xylitol, and the like.

The preferred non-ionic agents are sucrose, trehalose, mannitol, and glycerol, or a combination thereof. The more preferred non-ionic bulling agents are mannitol and sucrose, or a combination thereof.

The preferred compositions of the tonicity enhancing agents were 4-6% mannitol, 8-10% sucrose, or 8-10% trehalose. The more preferred compositions were 5% mannitol and 9% sucrose or trehalose.

It was noted that the negatively charged polysaccharides such as heparin and chondroitin sulfate at 0.5 mg/ml to 20 mg/ml did not help in preventing the aggregation of the conjugate at neutral pH.

### Example 5C

### Lower Ionic Strength is Preferred at Lower pH while Higher Ionic Strength is Preferred at High pH

The effects of increasing ionic strength with reagents such as NaCl, KCl, CaCl₂ facilitated conjugates aggregation at acidic pH. In particular, these salts at a concentration of 140 mM, pH 3.7, facilitated aggregation. At pH 5.5 to 7.5, the higher ionic strength is preferred over lower ionic strength in preventing the protein from aggregation. For example, 100 mM sodium phosphate, at pH 7.4 is better than its 10 mM concentration in preventing the aggregation.

The ionic strength of a solution is expressed as one-half the sum of CiZi² where C is the concentration, Z is the charge, and i represents ion.

Low ionic strength is preferred in low pH buffers while high ionic strength is preferred in high pH buffers. The preferred ionic strength in pH 3.0-4.0 buffers is lower than 10 mM and the preferred ionic strength in pH 5.5-7.5 buffers is 100-150 mM.

### Example 5D

### Effect of Surfactants

Non-ionic surfactants include polyoxyethylene sorbitol esters such as polysorbate 80 (Tween 80) and polysorbate 20 (Tween 20) and polyethylene glycol. Zwitterionic surfactant such as Zwittergent® was used to solubilize unmodified protein. The preferred non-ionic surfactants are polysorbate 80, polysorbate 20, and polyethylene glycol. Polysorbate 20 from Sigma prevented protein aggregation whereas Polysorbate 20 from Calbiochem and J. T. Baker did not.

### Example 5E

### Low Storage Temperature

Stability of the protein at different temperatures in various buffers, pHs, and excipients was investigated.

The stability of the protein decreases with elevated temperatures: −20° C., 4° C.>25° C.>37° C. The temperature of 37° C. was used to, accelerate the stability study. The preferred temperatures are −20° C. and 2-8° C.;

At 2-8° C., the conjugates were stable even in the presence of unfavorable components such as high salt (140 mM NaCl) or high pH (pH 7.4) for at least a few weeks. In low pH (3.0-4.0) and low ionic strength buffers, ten cycles of freeze-thaw from −80° C. to +20° C. caused about 2% aggregation. Each cycle of freeze-thaw from −80° C. to +37° C. caused about 3% aggregation.)

In pH 5.0-6.5 buffers, for example, 10 mM sodium acetate, pH 5.0, 150 mM NaCl, or 10 mM sodium phosphate, pH 6.5, 150 mM NaCl, in the presence or absence of Polysorbate 80, five cycles of freeze-thaw from −80° C. or −20° C. to +20° C. did not cause any aggregation or a loss of antiviral activity.

### Example 5F

### Protein Concentration

PEG-IFN-beta-1b concentrations between 0.1254 mg/ml were examined. The samples were incubated in 5% mannitol, 3 mM HAc (pH 3.7), 37° C. during the storage stability testing period.

The integrity of the conjugate was monitored by SEC-HPLC.

Lowering protein concentration lowered protein aggregation.

The preferred protein concentrations are between 3.0 mg/ml to 0.05 mg/ml.

Results for Storage of Different Concentrations

| Protein | Incubation | |
|---|---|---|
| concentration | at 37° C. | Aggregation |
| (mg/mL) | (day) | (%) |
| 0.050 | 17 | 3.3 |
| 0.10 | 17 | 2.0 |
| 0.125 | 1 | 0 |
| 0.25 | 1 | 0 |
| 0.50 | 1 | 0.3 |
| 1.0 | 1 | 0 |
| 2.0 | 1 | 0 |
| 4.0 | 1 | 5.7 |

### Example 5G

### Neutralization of Solution pH for Administration

Since acidic solution could cause skin irritation, it is noted that acidity can be neutralized by adding sodium phosphate solution or powder before administration. For example, when 1/10 volume of 10×PBS or powder with the same components was added to 1% mannitol, 3 mM HAc, pH 3.7, 0.30 mg/ml PEG-protein, the pH increased to 6.5. The sample after neutralization should be administered within 2 hrs at 25° C. or 20 hrs at 4° C. The effects of such pH neutralization on aggregation of the tested PEG-IFN-beta 1b was minimal, as summarized by the following table.

Effect of Neutralization on Aggregation*

| | | | Time after | |
|---|---|---|---|---|
| | | T | neutralization | Aggregation |
| Neutralization | pH | (° C.) | (hr) | (%) |
| no | 3.7 | 25 | 0 | 0 |
| yes | 6.5 | 25 | 0 | 0.5 |
| | | | 0.5 | 0.1 |
| | | | 1.0 | 2.7 |
| | | | 2.0 | 3.0 |
| | | | 18 | 13.8 |
| | | 4 | 0 | 0 |
| | | | 19 | 2.2 |

*Percent aggregation was analyzed by SEC HPLC

### Example 5H

### Antiviral Activity

Antiviral activity of PEG2-40k-IFN-beta-1b in 3 mM acetic acid, 5% mannitol, 0.3 mg/mL and various temperatures was examined on A549 cells/EMCV virus. Antiviral activity was expressed as percent of native IFN-beta-1b activity in side-by-side assays. The data in the table show that the conjugate was stable for at least eight months when stored at 4° C.

Stability and Antiviral Activity of PEG2-40k-IFN-beta-1b

| Temperature | Duration | Aggregation | Antiviral |
|---|---|---|---|
| (° C.) | (week) | (%) | activity (%) |
| −20 | 10 | 6 | n.d. |
| | 16 | 5 | 42 |
| +4 | 0 | 0 | 42 |
| | 8 | 4 | 42 |
| | 20 | 3 | 35 |
| | 24 | 2 | 27 |
| | 32 | 4 | 33 |
| +25 | 1 | 0 | n.d. |
| | 4 | 1 | n.d. |
| +37 | 1 | 0 | n.d. |
| | 4 | 2 | n.d. |

### Example 6

### Lyophilization

### Example 6a

### Addition of Mannitol in Lyophilization Buffer

This example confirms that inclusion of mannitol in the lyophilization buffer reduced aggregation and allowed for retained antiviral activity after reconstitution. The ratio of PEG2-40k-IFN-beta-1b to mannitol was 0.5-2.5% by weight. A 1% concentration of mannitol was preferred.

Addition of Mannitol in Lyophilization Buffers*

| | | Monomer (%) | | Antiviral activity (MU/mg | |
|---|---|---|---|---|---|
| | Mannitol | control, no | | control, no | |
| | (%) | lyophilization | lyophilization | lyophilization | lyophilization |
| IFN- | 0 | N/A | N/A | 16.97 | 4.71 |
| beta-1b | 0.1 | N/A | N/A | 15.36 | 12.03 |
| | 0.2 | N/A | N/A | 12.66 | 9.44 |
| | 1 | N/A | N/A | 10.28 | 10.39 |
| PEG2- | 0 | 92 | 91 | 6.07 | 4.2 |
| 40k- | 0.1 | 92 | 92 | 3.15 | 4.85 |
| IFN- | 0.2 | 92 | 92 | 4.21 | 4.23 |
| beta-1b | 1 | 93 | 92 | 5.47 | 4.27 |

*The protein concentration was 0.3-0.4 mg/ml and lyophilization buffers contained 3 mM HAc, pH 3.7 and mannitol as indicated. The reconstitution buffer was 3 mM HAc, pH 3.7.

### Example 6b

### Addition of Polysorbate to Lyophilization Buffers

The example confirms that the addition of polysorbate 80 to the lyophilization buffers allowed for retained antiviral activity of the tested PEG-IFN-beta 1b after reconstitution, as summarized by the following table.

Addition of Polysorbate 80 in Lyophilization Buffer*

Antiviral Activity
(MU/mg)**

| Polysorbate | control, no | |
|---|---|---|
| 80 (%) | lyophilization | lyophilization |
| 0 | 5.18 | 3.79 |
| 0.02 | 4.35 | 2.54 |
| 0.1 | 4.61 | 3.56 |
| 0.5 | 5.85 | 5.18 |

*The lyophilization buffer contained 5% mannitol, 3 mM HAc, and polysorbate 80 at the concentration indicated. The reconstitution buffer was 10 mM sodium phosphate, pH 7.4.
**Vero cell assay.

### Example 6c

### Effect of Reconstitution Buffer on PEG-Protein Aggregation

This example compares the efficacy of three different reconstitution buffers at pH 7.4 (10 mM sodium phosphate), pH 5.0 (10 mM sodium acetate), and 3.7 (3 mM acetic acid) for the incidence of aggregation in the tested PEG-IFN-beta 1b after lyophilization and reconstitution.

The lower the pH of the reconstitution buffers, the lower the amount of the aggregation. The preferred lyophilization buffer contained 0.1-2 mg/ml of the tested PEG-IFN-beta 1b, 0.1-5% mannitol, 3 mM HAc, pH 3.7, and 0.02-0.5% polysorbate 80 from J. T. Baker.

The lyophilized powder was reconstituted in a reconstitution buffer of 10 mM sodium acetate or sodium phosphate, pH 5.0-7.4, 0-140 mM NaCl.

Alternatively, the reconstitution buffer was 3 mM HAc, pH 3.7 to make 0.1:2 mg/ml PEG-protein. 10 mM Sodium phosphate, pH 7.4 was then added to neutralize the pH before administration. The effects of these two buffers on aggregation of the tested PEG-IFN-beta 1b is summarized by the following table.

Effect of Reconstitution Buffer*

| | | T | Time after | Aggregation |
|---|---|---|---|---|
| Buffer | pH | (° C.) | reconstitution (hr) | (%) |
| 3 mM Hac | 3.7 | 25 | 0 | 2.3 |
| | | | 6 | 1.8 |
| 3 mM HAc, then | 6.5 | 25 | 0 | 2.2 |
| neutralized with 10 mM | | | 2.5 | 3.4 |
| sodium phosphate, | | | 4.5 | 4.1 |
| pH 7.4 | | | | |
| 10 mM sodium | 6.5 | 25 | 0 | 4.3 |
| phosphate, pH 6.5, | | | 7 | 5.4 |
| 120 mM NaCl | | | | |

*Percent aggregation was analyzed by SEC HPLC

### Conclusions

From the foregoing the characteristics of the inventive IFN-beta 1b polymer conjugate, in solution, can be summarized.

The preferred buffers (at −20° C., −80° C., or +4° C.) are composed of glycine-HCl, citrate, acetate, or aspartate with pH between 3.0 and 5.0 and the concentration between 5-10 mM. The buffer ionic strength of the buffers described above, was preferably lower than 10 mM. Also preferred are the glycine-HCl, citrate, acetate, aspartate, phosphate, and carbonate buffers with a pH ranging from 3-8. Preferably, acidic buffers are neutralized with sodium phosphate before administration.

Preferred carbohydrate excipients include, mannitol, sorbitol, sucrose, trehalose, and glycerol, and/or a combination thereof. When the buffer is employed as a lyophilization buffer, the preferred carbohydrate excipients include mannitol, sucrose, or trehalose, or a combination thereof, in a concentration ranging from 0.1-5% (w/v). Preferred surfactants employed as excipients include polysorbate 80, polysorbate 20, and/or polyethylene glycol. When the buffer is employed as a lyophilization buffer, the preferred surfactant excipients include polysorbate 80 or polysorbate 20 at 0.002-0.5% (w/v).

The preferred reconstitution buffer was sodium acetate or sodium phosphate, pH 5.0-7.4, plus NaCl added until isotonicity was reached.

Other preferred reconstitution buffers were glycine-HCl, citrate, acetate, or aspartate prepared with a pH ranging from 3.0-4.0, followed by neutralization with sodium acetate or sodium phosphate to a final pH ranging from 5.0-7.4 for administration.

### Example 7

### Immunogenicity and In Vitro Stability

### Experimental Design:

Sprague Dawley (Harlan) rats weighing 150-300 g (three in a group) were administered intramuscularly or subcutaneously with native IFN-beta-1b or PEG-IFN-beta-1b conjugates at 0.1 mg/kg, once per week for 3-6 weeks. The plasma samples were collected seven days after the previous injection and right before the next injection.

### Assay Design:

The antibodies produced against IFN-beta-1b or PEG-beta-1b conjugates were analyzed by direct ELISA where the capture reagent was IFN-beta-1b and detection antibody was horse radish peroxidase conjugated rabbit against rat IgG. Results are listed in the following table.

Analysis of Rat Anti hIFN-beta-1b Antibodies by ELISA (μg/ml)*

| Antigen | Week 1 | Week 2 | Week 3 | Week 4 | Week 6 |
|---|---|---|---|---|---|
| IFN-beta-1b | 4.89 ± 4.42 | 119.8 ± 84.77 | 161.87 ± 97.82 | 305.37 ± 28.88 | 233.16 ± 55.75 |
| ALD-PEG-40k | 6.47 ± 0.35 | 3.46 ± 1.82 | 9.70 ± 8.95 | 13.03 ± 6.56 | 7.20 ± 2.62 |
| PEG2-40k | 3.52 ± 2.74 | 7.29 ± 1.66 | 7.93 ± 0.03 | 12.59 ± 0.66 | 7.53 ± 1.18 |
| PEG-U-Ala-40k | 7.49 ± 1.73 | 3.65 ± 3.26 | 4.42 ± 3.56 | 8.03 ± 5.20 | 5.02 ± 1.93 |
| Di PEG-20k | 6.07 ± 2.42 | 4.98 ± 4.47 | 3.54 ± 0.13 | 8.44 ± 5.91 | 4.40 ± 2.28 |

*Mouse anti h IFN-beta monoclonal antibody (R&D, #21405-1, clone#MMHB-3, IgG1, kappa) was used as standard.

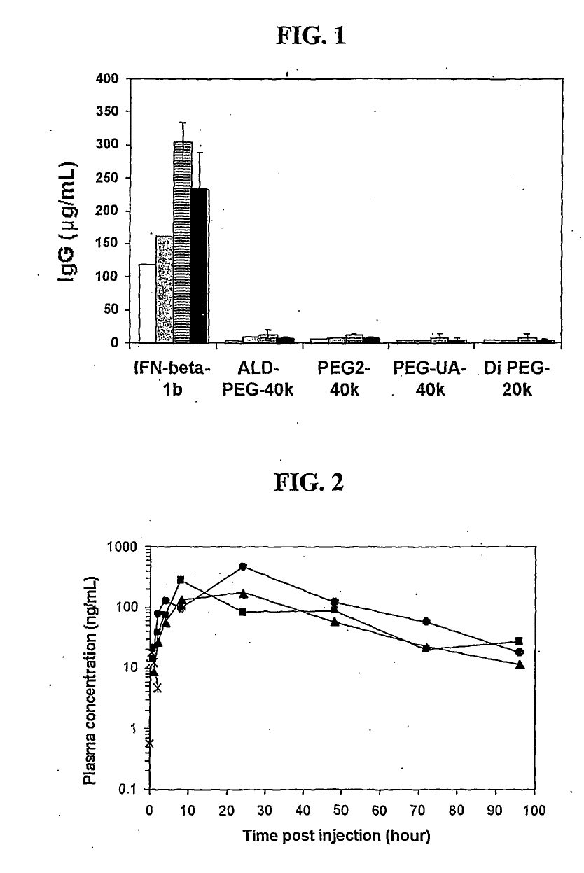

See also FIG. 1.

### Conclusions

From the foregoing, it is concluded that the inventive IFN-beta 1b polymer conjugate provides a number of advantages, including:
PEGylation greatly reduced immunogenicity of the protein.
Immunogenicity (IgG titers) of IFN-beta-1b was reduced by 94-98% after PEGylation with mono PEG40k and di PEG-20k.
The rat immune system was more tolerant of PEG-protein than the native protein, as confirmed by the determination that was no significant increase of antibodies from first to sixth dose.
The antibodies were neutralizing antibodies when analyzed by antiviral bioassays.
There was no increased production of antibodies after 4 doses with IM administration.

It was discovered that the PEG-IFN-beta compounds were more resistant toward proteases in mouse kidney and liver extracts upon PEGylation. The half life of IFN-beta-1b increased by 6 fold in both kidney and liver extracts after PEGylation. The stability was analyzed by ELISA.

It was further discovered that the PEG-IFN-beta compounds were more resistant to oxidation by hydrogen peroxide after PEGylation.

### Example 8

### Enhanced Pharmacokinetic Profiles

Pharmacokinetic Parameters in Rats

| | | Dose | Half-life | AUC | Tmax | Cmax |
|---|---|---|---|---|---|---|
| Compound | Rt | (mg/kg) | (hr) | (hr · u/mL) | (hr) | (u/ml) |
| IFN-beta-1b | IV | 0.6 | 1.08 | 26210 | NA | |
| PEG2-40k | IV | 0.6 | 9.43 | 751328 | NA | |
| PEG-U-Ala- | IV | 0.6 | 12.0 | 687389 | NA | |
| 40k | | | | | | |
| IFN-beta-1b | SC | 0.6 | 2.43 | 323.9 | 1.0 | 95.3 |
| PEG2-40k | SC | 0.6 | 23.8 | 72014 | 24 | 1829.2 |
| PEG-U-Ala- | SC | 0.6 | 18.1 | 42938 | 48 | 798.3 |
| 40k | | | | | | |
| IFN-beta-1b | IM | 0.6 | 2.29 | 805.1 | 0.5 | 135.8 |
| PEG2-40k | IM | 0.6 | 15.2 | 164920 | 8.0 | 4908.6 |
| PEG-U-Ala- | IM | 0.6 | 14.4 | 76782 | 8.0 | 2989.8 |
| 40k | | | | | | |

*By Vero cell assay

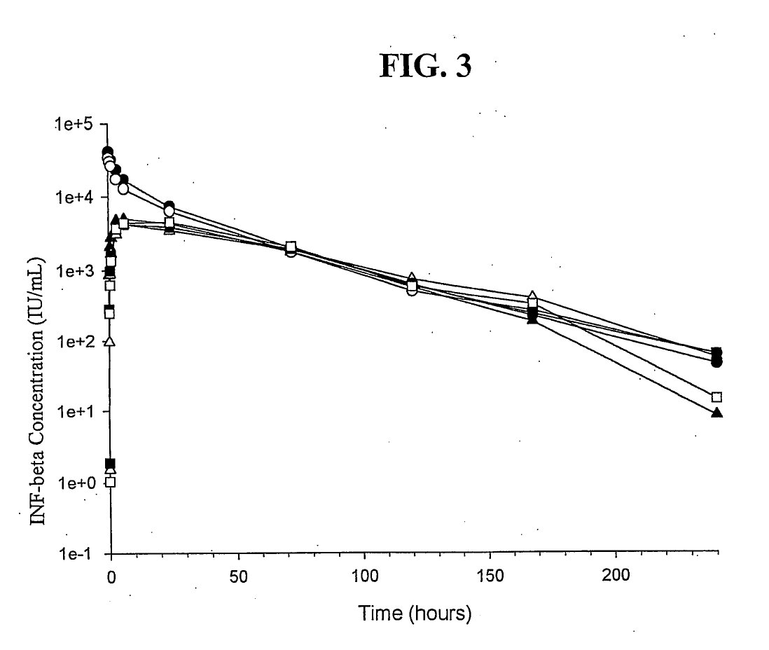

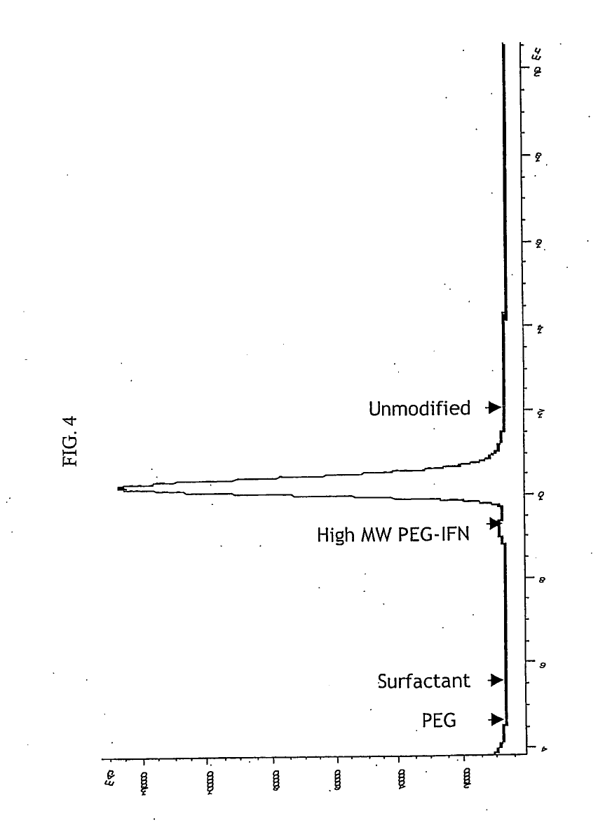

See also FIG. 2.

The results of the pharmacokinetic studies can be summarized as follows.
The AUC of IFN-beta-1b was enhanced by more than 90 fold by SC or IM administration and clearance rate was prolonged by more than 80 fold after mono PEGylation with PEG-40k.
The bioavailability of PEGylated IFN-beta-1b was better when administered by the IM route than when administered by the SC route, in both mice and rats.
The bioavailability of the PEGylated IFN-beta-1b was better than the native IFN-beta.

Bioavailability of IFN-beta-1b and PEG-IFN-beta-1b
Conjugate in Mice and Rats

| | | Dose | | Bioavailability | |
|---|---|---|---|---|---|
| | | (mg/kg) | | (%)* | |
| Compound | Species | SC | IM | SC | IM |
| IFN-beta-1b | mouse | 0.2 | 0.1 | 15 | 30 |
| PEG2-40k-IFN-beta- | | 0.2 | 0.1 | 22 | 42 |
| 1b | | | | | |
| IFN-beta-1b | rat | 0.6 | 0.6 | 0.96 | 2.0 |
| PEG2-40k-IFN-beta- | | 0.6 | 0.6 | 8.9 | 34 |
| 1b | | | | | |

*Average numbers from Vero and A549 antiviral (EMC) cell assays.

### Example 9

### Pharmacokinetic Profiles in Monkey

This example provides the following information.

The serum kinetics of EZ-2046 PEG-IFN-beta in Cynomolgus monkeys after administering the EZ-2046 polymer conjugate. EZ-2046 is an amide-linked conjugate of recombinant IFN-beta-1b with a single branched 40 kDa PEG.

The effect of different routes of administration on pharmacokinetics and pharmacodynamics

The bioavailability EZ-2046 following SC or IM administration;

The relationship between EZ-2046 administration and the pharmacodynamic marker neopterin.

### Materials and Methods

Male and female Cynomolgus monkeys were single-dosed by intravenous (“IV”), subcutaneous (“SC”), or intramuscular (“IM”) administration with EZ-2046 PEG-IFN-beta) at a dose level of 15 μg/kg or 480,000 μl/kg IFN-beta equivalents (IFN-beta specific activity 32MIU/mg). In vitro antiviral activity of the conjugate indicated that 32%-34% of the native activity of IFN-bet was retained, therefore, the activity adjusted dose of the conjugate was approximately 160,000 IU/kg. Since these methods can not differentiate free IFN-beta from pegylated IFN-beta, IFN-beta equivalence, including both forms of drug, are actually measured. For simplicity, IFN-beta and IFN-beta equivalent are interchangeable in this report. Pharmacokinetic parameters were assessed for EZ-2046 by ELISA or bioactivity analysis of serum.

The pharmacodynamic effects were evaluated using an ELISA assay to examine plasma neopterin levels as a marker for EZ-2046 biological activity in vivo. Neopterin is a pteridine derivative derived from guanosine triphosphate and is produced by lymphocytes and/or macrophages in response to stimulation of the immune system. It is a well known biomarker for in vivo interferon bioactivity in primates and humans.

EZ-2046 maximum plasma concentrations (“Cₘₐₓ”), plasma terminal elimination half-life (“t_{1/2}”), area under the serum concentration-time curve for the period of 0 to infinity (“AUC_{0-□}”), and bioavailability were determined using either one compartment (SC, IM) or two compartment (IV) first order pharmacokinetic models.

Neopterin E₀ (serum baseline level), T_{lag} (lag time following administration), Tₘₐₓ (Time to reach maximum concentration), Eₘₐₓ (net effect maximum), K10_HL (the rate neopterin leaves the serum compartment half-life, and AUC (area under the curve of serum concentration verses time), were determined using a single compartment first order uptake, lag, first order elimination model.

### Sample Collection

Blood samples were collected from three (3) animals/sex/group at 10 and 30 minutes and 1, 3, 6, 24, 72, 120, 168, and 240 hours after injection. Samples were collected into tubes and allowed to clot for 20 minutes at room temperature prior to being placed on ice in an upright position. After serum separation, serum was distributed into 5 tubes and frozen at approximately −70° C. (or lower) within 2 hours after collection until analysis. Animals were bled prior to dosing for baseline levels.

### Determination of Serum

Two different methods were employed to determine serum EZ-2046. In method A, the concentration of serum EZ-2046 was determined by an ELISA assay. In method B, the bioactivity of serum EZ-2046 was determined by means of an antiviral assay employing A549 cells challenged with Encephalomyocarditis (murine) virus.

ELISA Quantitative Determination of Serum IFN-beta

Serum IFN-β concentrations were determined using a commercially available one-step sandwich ELISA assay kit (Immuno-Biological Laboratories, Cat # MG53221). The assay was performed as described by manufacture.

1. Equipment

(a) Polypropylene microtiter tubes. Catalog no. 29442-608, VWR, S. Plainfield, N.J. or equivalent source.

(b) Precision repeating pipettors were employed to deliver 100 μl and 1000 μL, 100 μL fixed volume pipette and 100 μL adjustable pipette. Eppendorff or equivalent, VWR, S. Plainfield, N.J. 07080 or equivalent.

(c) Absorbent Paper.

(d) Molecular Device Versamax plate reader, Sunnyvale, Calif.

(e) Automated Plate Shaker: Lab-Line Instruments Inc., IL, USA.

(f) Bio-Tek Plate washer, ELx405, Winooski, Vt.

2. Materials

(a) Human Interferon-beta ELISA Kit: Immuno-biological Laboratories, Cat No. MG 53221, Lot # GL40502, Minneapolis, Minn.

3. Preparation of Buffers/Solutions

(a) Wash solution.

(i) Diluted 50 mL Wash solution concentrate (Bottle 4) to 450 mL with distilled water. Use at room temperature prior to use. Store at 4° C.

(a) Dilution Buffer.
(i) Ready to use. “Bottle 5.” Use and store at 4° C.

(b) Enzyme-labeled antibody Solution
(i) Dissolved vial (#2) of HRP-labeled mouse monoclonal antibody to IFN-beta Hu), Fab′ in 6 mL dilution buffer. Used and stored at 4° C.

(a) Substrate Solution
(i) Prior to use combined 10 mL “Substrate “A” (vial 6) with 0.5 mL “Substrate B” (vial 7). Used immediately at room temperature.

(a) Stop Reagent

Stop solution (Bottle 8) Ready to use. Used at room temperature.

4. Calibration Standards

(a) Reconstituted lyophilized IFN-beta Hu) standard with 1 mL ice cold water with gentle agitation to obtain a working solution with a concentration (IU/mL) described on the vial label. Diluted the working solution with ice cold Dilution Buffer to make standards at 200, 100, 50, 20, 10, 5, and 2.5 IU/mL. Dilution buffer was used as a standard solution for 0 IU/mL Dilutions were made on ice with gentle mixing.

5. Immunoassay Procedure

(a) Allowed antibody coated microwell assay plate to come to room temperature prior to use.

(b) Added 400 μL of washing buffer per well to assay plate. Completely removed the buffer by aspiration.

(c) Added 50 μL enzyme-labeled antibody per well.

(d) Added 100 μL standards or test samples per well in duplicate using grid map.

(e) Sealed plate and incubate 2 hours at room temperature (20-30° C.) with orbital shaking (350-450 rpm).

(f) Removed solutions and wash plate 4 times with 1 minute incubation between wash intervals.

(g) Added 100 μL substrate solution per well.

(h) Sealed plate and incubate 30 minutes at room temperature with shaking

(i) Added 100 μL Stop solution

(j) Read plate at 450 nm with 570 nm reference

(k) The serum concentrations were determined from the calibration curve generated using a 4-parametric curve fit method.

### B. Bioactivity Assay for IFN-beta in Cynomolgus Monkey Serum

1. Equipment

(a) 96 well polystyrene tissue culture plate: Catalog no. 29442-054, VWR, S. Plainfield, N.J., 07080 or equivalent source.

(b) Polypropylene Microtiter tubes, Catalog no. 29442-608, VWR, S. Plainfield, N.J. or equivalent source.

(c) Multichannel 250 μL and 1000 μL adjustable pipettes. Finnpipette or equivalent, VWR, S. Plaifield, N.J., 07080.

(c) Sterile paper towel.

(d) Incubator, CO₂, humidified Form a, USA.

(e) Molecular Device plate reader model Versamax, Sunnyvale, Calif.

2. Materials

(a) IFN-beta calibration standards, Catalog I-4151, Sigma, Lot #082K16781, store 2-8° C.

(b) Ham's F12K medium, Catalog #30-2004, ATCC, Lot #3000144, store 2-8° C.

(c) MEM, Catalog #20-2003, ATCC, Lot #3000302.

(d) Fetal bovine serum, Catalog no. SH30071, Hyclone, Logan, Utah.

(e) Penicillin and Streptomycin, Catalog #15140-122, Gibco, USA.

(f) Phosphate buffered saline (PBS), Catalog #17-516F, Lot #01104281, BioWhittaker, USA

(g) A549 Cells, Catalog # CCL-185, ATCC

(h) Encephalomyocarditis Murine Virus (EMCV), Enzon Pharmaceutical Lot # V6, produced in Vero cells (CCL-81, ATCC) from EMCV, Catalog number VR-129B, ATCC.

(i) A solution of 3-(4,5-dimethyl-thiazol-2-yl)-2,5-diphenyl tetrazolium bromide (“CTI”), Catalog # G4102, Lot 18264601, Promega, USA.

(3) Solubilization/Stop solution, Catalog # G4101, Lot #178861, Promega, USA.

3. Bioassay Procedure

The bioactivity of serum EZ-2046 was determined by examining its antiviral activity, i.e., the amount of protection provided by EZ-2046 to A549 cells challenged with Encephalomyocarditis virus (EMCV) infection. Serial dilutions of serum samples or IFN-β standard were added in triplicate to wells of a 96-well plate. A549 cells (10⁴/well) in Ham's-F12K containing 10% fetal bovine serum (BS) were added to the wells of the plate and incubated overnight at 37° C. in 5% CO₂. The growth medium was removed and 50 μL/well EMCV (1.1×10⁵ PFU/mL) added to the plate and incubated for 2-hours at 37° C. under 5% CO₂. The virus inoculum was removed and the cells fed Ham's-F12K containing 5% FBS. Plates were incubated for 40 hours at 37° C. in 5% CO₂. Fifteen microliters of MTT solution (Promega Corporation) was added to each well of the plate and the plate incubated for 4 hours at 37° C. with 5% CO₂. The wells were solubilized with 100 μL solubilization/stop solution (Promega) over night at room temperature in the dark. The optical density of the wells were determined at 570 nm with a 630 nm reference and the serum concentrations of the samples were determined from the standard calibration curve generated using a 4-parameter fit.

### Determination of Serum Neopterin by

### Immunassay As a Biomarker for IFN-Beta Activity

Serum neopterin concentrations were determined using a commercially available competitive ELISA assay kit (Immuno-Biological Laboratories, Cat # RE59321). The assay was performed as described by manufacture, as follows.

### 1. Equipment

a. Polypropylene microtiter tubes. Catalog no. 29442-608, VWR, S. Plainfield, N.J. or equivalent source.
b. Precision repeating pipettors to deliver 100 μl and 1000 μL, 100 μL fixed volume pipette and 100 μL adjustable pipette. Eppendorff or equivalent, VWR, S. Plainfield, N.J. 07080 or equivalent
c. Absorbent Paper
d. Molecular Device Versamax plate reader, Sunnyvale, Calif.
e. Automated Plate Shaker: Lab-Line Instruments Inc., IL, USA
f. Bio-Tek Plate washer, ELx405, Winooski, Vt.

2. Materials

Neopterin (Hu)□ELISA Kit: IBL Immuno-Biological Laboratories, Cat No. RE59321, Lot # ENO187, Minneapolis, Minn.

### 3. Preparation of Buffers/Solutions

a. Wash solution
Diluted 50 mL of Wash solution concentrate to 450 mL with distilled water. Used at room temperature prior to use. Stored at 4° C. for up to 1 month.

b. Assay Buffer
Ready to use. Use at room temperature and store at 4° C.

c. Enzyme-labeled antibody Solution
a Add 25 μL antibody concentrate in 5 mL assay buffer (1:201). Use at room temperature and store at 4° C. for 24 hours protected from light.

d. Substrate Solution
Prior to use add 300 μL TMB Substrate to 9 mL Substrate buffer. Use immediately at room temperature. Store at 4° C. for up to 48 hours.

e. Stop Reagent
Stop solution is ready to use. Use at room temperature.

### 4. Calibration Standards

a. Calibration standards are ready for use containing neopterin in phosphate buffer with stabilizers. Assay buffer is used as the zero standard.

| Standard | A | B | C | D | E | F |
|---|---|---|---|---|---|---|
| nmol/L | 0 | 1.35 | 4.0 | 12.0 | 37.0 | 111 |
| ng/mL | 0 | 0.33 | 1.0 | 3.0 | 9.0 | 28 |

b. Control sera for quality control are provided ready for use.

5. Immunoassay Procedure
c. Antibody coated microwell assay plates were allowed to come to room temperature prior to use.
d. Added 10 μL of each standard, control or monkey serum sample in duplicate to wells of microassay plate using grid map.
e. Added 100 μL Enzyme Conjugate per well.
f. Added 50 μL Neopterin antiserum
g. Sealed plate with black foil and incubate in the dark for 90 minutes at room temperature (20-30° C.) with orbital shaking (350450 rpm).
h. Removed solutions and wash plate 4 times with 1 minute incubation between wash intervals.
i. Added 200 μL substrate solution per well.
j. Sealed plate and incubate 10 minutes at room temperature with shaking
k. Added 100 μL Stop solution
l. Read plate at 450 nm with 650 mm reference
m. The serum concentrations were determined from the calibration curve generated using a 4-parametic curve fit method.

### Software

Descriptive statistics (mean and standard deviation or SD) and compartmental and non-compartmental analyses were performed using WinNonlin Professional version 4.1 (Pharsight Corporation). Calculations of serum concentrations for ELISA and bioassays were performed using Microsoft® Excel 2002. Graphical presentations were carried out using SPSS® SigmaPlot version 8.0. Comparative statistics were performed using SigmaStat version 3.01.

### Results

Tabulated EZ-2046 Concentration-time Profile Determined by ELISA
Mean Pharmacokinetic Parameters for EZ-2046 determined by ELISA

| | | | | | | | Plasma Terminal | | | | | | | | |
|---|---|---|---|---|---|---|---|---|---|---|---|---|---|---|---|
| | | | | | | | Elimination | | | | | | | | |
| | Tₘₐₓ | | | Cₘₐₓ | | | Half-life (t) | | | AUC_{0- □} | | | Bioavailability | | |
| | (h) | | | (IU/mL) | | | (h) | | | (MIU^{□}h/mL) | | | (%) | | |
| Rt | M | F | All | M | F | All | M | F | All | M | F | All | M | F | All |
| SC | 11.6 | 14.0 | 12.8 | 5500 | 4800 | 5200 | 32.0 | 45.9 | 39.0 | 0.35 | 0.34 | 0.34 | 69.3 | 61.2 | 65.0 |
| | (7.1) | (9.0) | (7.4) | (2200) | (1800) | (1900) | (5.2) | (18.5) | (14.3) | (0.13) | (0.04) | (0.09) | | | |
| IM | 5.4 | 8.7 | 7.1 | 4300 | 5300 | 4800 | 47.7 | 41.1 | 44.4 | 0.34 | 0.34 | 0.34 | 67.1 | 61.4 | 64.1 |
| | (4.5) | (0.3) | (3.4) | (500) | (1400) | (1100) | (14.5) | (7.9) | (11.1) | (0.09) | (0.07) | (0.07) | | | |
| IV | | | | 35100 | 41500 | 38300 | 22.7 | 18.5 | 20.6 | 0.50 | 0.56 | 0.53 | — | — | — |
| | | | | (3000) | (3200) | (4500) | (5.5) | (6.4) | (5.8) | (0.11) | (0.14) | (0.12) | | | |

“Rt” is the route of administration;
“SC” is subcutaneous;
“IM” is intramuscular,
“IV” is intravenous,
“F” is female,
“M” is male.

### Conclusions:

Mean EZ-2046 serum concentration values were similar in male and female, resulting in similar EZ-2046 concentration-time profiles.
The EZ-2046 pharmacokinetic values for monkeys receiving subcutaneous and intramuscular administrations were similar to each other:
Intravenous administered monkey had a 6 to 8 fold higher Cₘₐₓ, 1.4 to 2.5 fold slower terminal elimination and 1.4 to 1.6 fold larger AUC compared to SC and IM administration.
Bioavailability of EZ-2046 following SC and IM administration was approximately 65%.
EZ-2046 was measurable in serum 168 hours following administration.

### B. EZ-2046 Concentration-Time Profile Determined by BioActivity Assay

ELISA analysis provides a protein concentration profile of EZ-2046 kinetics. It was also used to determine the kinetics of bioactive EZ-2046. This data is summarized as the ratio between ELISA pharmacokinetic parameter estimates and the bioactivity assay pharmacokinetic parameter.

Ratio of EZ-2046 ELISA Pharmacokinetic
Estimates over EZ-2046 Bioactivity Assay Estimates

| | C_{max ELISA/} | | | t_{ELISA}/ | | | AUC_{0- □ ELISA/} | | | | | |
|---|---|---|---|---|---|---|---|---|---|---|---|---|
| | C_{max Bioassay} | | | t_{Bioassay} | | | AUC_{0- □ Bioassay} | | | Bioavailability | | |
| Rt | M | F | All | M | F | All | M | F | All | M | F | All |
| SC | 14.7 | 11.4 | 13.1 | 2.6 | 3.2 | 3.0 | 24.0 | 28.3 | 26.1 | 1.8 | 1.8 | 1.8 |
| IM | 15.3 | 5.8 | 8.1 | 3.1 | 2.6 | 2.9 | 30.1 | 15.1 | 20.1 | 2.2 | 1.0 | 1.4 |
| IV | 1.6 | 6.3 | 2.8 | 4.5 | 2.5 | 3.2 | 13.5 | 15.8 | 14.7 | — | — | — |

“Rt” is the route of administration;
“SC” is subcutaneous;
“IM” is intramuscular,
“IV” is intravenous,
“F” is female,
“M” is male.

### Conclusions:

The EZ-2046 bioactivity assay showed an 8.1 to 13.1 fold decrease in mean EZ-2046 Cₘₐₓ activity compared to the Cₘₐₓ concentration determined by ELISA following intramuscular and subcutaneous administration of EZ-2046 and a 2.8 fold decrease following intravenous administration.
All routes of administration showed similar bioactivity decreases in terminal serum elimination half-life (t_{1/2}) ranging from 2.9 to 3.2 fold compared to ELISA determined serum elimination half-life.
The EZ-2046 bioactivity AUC, likewise, decreased 14.7, 20.1 and 26.1 fold following IV, SC, and IM administration compared to ELISA values.
Subcutaneous and intramuscular administered EZ-2046 showed a similar 1.4 to 1.8 fold decrease in bioavailability by the IFN-beta bioactivity assay compared to ELISA estimates.

### C. EZ2046 Pharmacodynamic Effect Determined by Neopterin Synthesis

The pharmacologic effect of EZ-2046 administration was determined by examining the serum neopterin response using the methods describe supra.

Mean Pharmacodynamic Parameters for Neopterin

| | | | | | | | | | | Effect | | | | | |
|---|---|---|---|---|---|---|---|---|---|---|---|---|---|---|---|
| | | | | | | | | | | Half-life | | | | | |
| | T_{lag} | | | Tₘₐₓ | | | Eₘₐₓ* | | | (K10_HL) | | | AUC | | |
| | (h) | | | (h) | | | (ng/mL) | | | (h) | | | (h□ng/mL) | | |
| Rt | M | F | All | M | F | All | M | F | All | M | F | All | M | F | All |
| SC | 4.5 | 4.3 | 4.4 | 39.3 | 29.7 | 34.0 | 3.2 | 3.1 | 3.2 | 33.7 | 60.2 | 47.0 | 314 | 351 | 332 |
| | (1.8) | (1.5) | (1.9) | (9.8) | (15.8) | (12.7) | (1.0) | (0.9) | (0.9) | (7.6) | (36.9) | (27.9) | (84) | (30) | (60) |
| IM | 6.9 | 4.8 | 5.8 | 27.2 | 31.0 | 29.1 | 1.7 | 2.4 | 2.0 | 63.5 | 57.8 | 60.7 | 197 | 274 | 236 |
| | (2.7) | (1.2) | (2.2) | (6.5) | (5.5) | (5.8) | (0.4) | (0.6) | (0.6) | (40.3) | (19.2) | (28.4) | (95) | (79) | (89) |
| IV | 6.9 | 4.8 | 5.8 | 27.3 | 28.8 | 28.1 | 3.1 | 3.1 | 3.2 | 43.9 | 42.4 | 43.2 | 310 | 284 | 297 |
| | (2.7) | (1.2) | (2.2) | (12.0) | (9.6) | (9.8) | (0.3) | (0.3) | (0.4) | (10.9) | (3.1) | (7.2) | (109) | (50) | (77) |

E₀: mean serum baseline for neopterin was 1.4 ng/mL

### Conclusions:

Irrespective of route of administration the pharmacodynamic parameters for neopterin synthesis were similar.
Neopterin synthesis occurred 4 to 7 hours following administration of EZ-2046 (T_{lag}).
The time to the maximal effect (Tₘₐₓ) ranged from 28 to 34 hours.
The maximum effect of the IFN-beta 1b (Eₘₐₓ) ranged from 2.0 to 3.2 ng/mL above baseline neopterin levels of 1.4 ng/mL.
The serum neopterin effect diminished with an elimination half-life ranging from 43.2 to 60.7 hours.
Neopterin exposure above background levels ranged from 236 to 332 h□ng/mL.

### Additional Conclusions:

Following administration of PEG-IFN-β conjugate EZ-2046 to Cynomolgus monkeys the pharmacokinetic and pharmacodynamic parameters for IFN-β and neopterin were similar between genders.
Subcutaneous and intramuscular administration of EZ-2046 showed comparable Cₘₐₓ, terminal serum elimination half-life (t_{1/2}), AUC, and bioavailability. The SC and IM EZ-2046 serum elimination half-life was approximately 2-fold slower compared to intravenous administration.

The bioactivity of EZ-2046 showed a 3 fold decrease in Cₘₐₓ following administration compared to ELISA values, most likely due to the lower specific activity of the pegylated IFN-beta as compared to the unconjugated drug.
The neopterin response was similar irrespective of route of administration of EZ-2046
Neopterin increased slowly two-fold above baseline post EZ-2046 administration and the neopterin response diminished slowly and was still detectable a week after EZ-2046 administration.

We claim:

1. A composition comprising:
a) interferon conjugated to a polyalkylene oxide polymer having a molecular weight of at least about 12 kDa; and optionally
b) a surfactant;
c) an excipient, and
d) a buffer
wherein the pH range of the solution is from about 3 to about 11.

2. The composition of claim 1 wherein the interferon is interferon-beta 1b.

3. The composition of claim 1 wherein the surfactant is selected from the group consisting of polyoxyethylene sorbitol esters and polyethylene glycol.

4. The composition of claim 1 wherein the pH range is from about 2.5 to about 8.5.

5. The composition of claim 1 wherein the pH range is from about 3.0 to about 5.0.

6. The composition of claim 1 wherein the pH range is from about 3.0 to about 4.0.

7. The composition of claim 1 wherein the buffer is selected from the group consisting of Glycine-HCl, acetic acid, sodium acetate, sodium aspartate, sodium citrate, sodium phosphate and sodium succinate.

8. The composition of claim 1 wherein the buffer is selected from sodium acetate, sodium citrate and glycine HCl.

9. The composition of claim 1 wherein the buffer has an ionic strength of about 10 mM.

10. The composition of claim 1 wherein the buffer is present in a concentration of from about 3 mM to about 10 mM.

11. The composition of claim 1 wherein the excipient is non-ionic and is selected from the group consisting of, monosaccharides, disaccharides, and alditols.

12. The composition of claim 7 wherein the excipient is selected from the group consisting of glucose, ribose, galactose, D-mannose, sorbose, fructose, xylulose, sucrose, maltose, lactose, trehalose, raffinose, maltodextrins, dextrans, glycerol, sorbitol, mannitol, and xylitol.

13. The composition of claim 8 wherein the excipient is selected from the group consisting of sucrose, trehalose, mannitol and glycerol or a combination thereof.

14. The composition of claim 9 wherein the excipient is selected from the group consisting of mannitol and sucrose or a combination thereof.

15. The composition of claim 1 wherein the surfactant is non-ionic and is selected from the group consisting of polysorbate 80, polysorbate 20, and polyethylene glycol.

16. The composition of claim 1 wherein the polyalkylene oxide polymer is linear or branched.

17. The composition of claim 1 wherein the linear polyalkylene oxide polymer is of the formula:
A-O—(CH₂CH₂O)ₓ—
-A-O—(CH₂CH₂O)ₓ—CH₂C(O)—O—,
A-O—(CH₂CH₂O)ₓ—CH₂CH₂NR₇—,
A-O—(CH₂CH₂O)ₓ—CHₓCH₂SH,
—O—C(O)CH₂—O—(CH₂CH₂O)ₓ—CH₂C(O)—O—,
—NR₇CH₂CH₂—O—(CH₂CH₂O)ₓ—CH₂CH₂NR₇—,
—SHCH₂CH₂—O—(CH₂CH₂O)ₓ—CH₂CH₂SH—,
wherein
A is a capping group;
R₇ is selected from hydrogen, C₁₋₆alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ alkenyls, C₃₋₁₂ branched alkenyls, C₁₋₄alkynyls, C₁₋₂ branched alkynyls, C₁₋₆ heteroalkyls, substituted C₁₋₆ hetero-alkyls, C₁₋₆ alkoxyalkyl, phenoxyalkyl and C₁₋₆ heteroalkoxys, and
x is the degree of polymerization.

18. The composition of claim 5 where in said capping group is selected from the group consisting of OH, CO₂H, NH₂, SH, and C₁₋₆ alkyl moieties.

19. The composition of claim 1 wherein the branched polyalkylene oxide polymer is selected from the group consisting of: 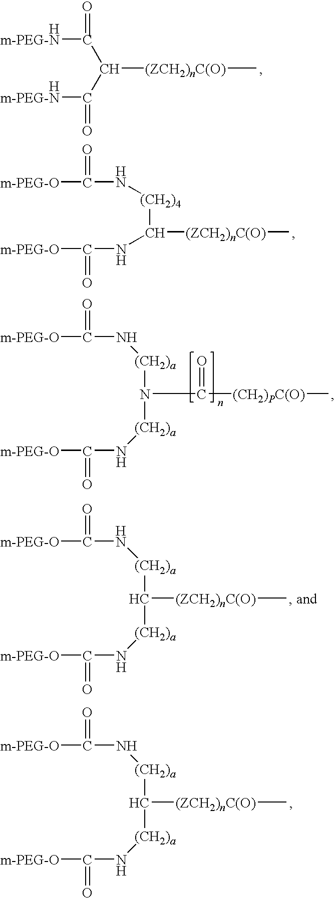
wherein:
(a) is an integer of from about 1 to about 5;
Z is O, NR₈, S, SO or SO₂; where R₈ is H, C₁₋₈alkyl, C₁₋₈ branched alkyl, C₁₋₈ substituted alkyl, aryl or aralkyl;
(x) is the degree of polymerization;
(n) is 0 or 1;
(p) is a positive integer, preferably from about 1 to about 6;
m-PEG is CH3-O—(CH2CH2O)ₓ—, and
The ligand is interferon-beta 1b.

20. The composition of claim 1 wherein the interferon-beta 1b comprises the amino acid sequence of SEQ ID NO:1.

21. The composition of claim 20 wherein the interferon-beta 1b is conjugated to a polyalkylene oxide polymer selected from the group selected from:
A-O—(CH₂CH₂O)_{X}—
A-O—(CH₂CH₂O)ₓ—CH₂C(O)—O—,
A-O—(CH₂CH₂O)ₓ—CH₂CH₂NR₇—,
A-O—(CH₂CH₂O)ₓ—CH₂CH₂SH,

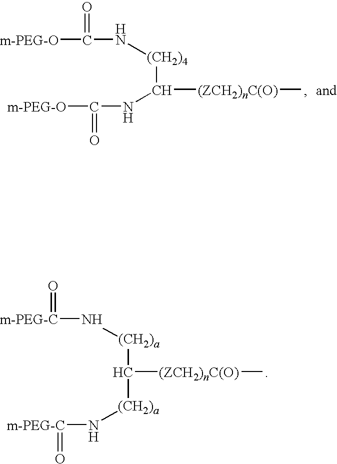

22. The composition of claim 21 wherein the molecular weight of the polyalkylene oxide polymer ranges from about 12 kDa to about 60 kDa.

23. The composition of claim 21 wherein the molecular weight of the polyalkylene oxide polymer is about 30 kDa.

24. The composition of claim 21 wherein the molecular weight of the polyalkylene oxide polymer is about 40 kDa.

25. The composition of claim 1 wherein the polyalkylene oxide polymer is conjugated to the interferon-beta 1b by a linkage selected from the group consisting of urethane, secondary amine, amide, or thioether.

26. The composition of claim 1 wherein the interferon-beta 1b is conjugated to a polyalkylene oxide polymer via the alpha-amino-terminal of the interferon-beta 1b.

27. The composition of claim 1 wherein the interferon-beta 1b is conjugated to a polyalkylene oxide polymer via an epsilon amino group of a Lys of the interferon-beta 1b.

28. The composition of claim 1 wherein the interferon conjugate is present at a concentration of from about 0.01 mg/ml to about 4 mg/ml.

29. The composition of claim 28 wherein the interferon conjugate is present at a concentration of from about 0.05 mg/ml to about 3 mg/ml.

30. A composition comprising:
a) 0.05 to 3.0 mg/ml of interferon beta 1b conjugated to a polyalkylene oxide polymer having a molecular weight of at least about 12 kDa,
b) 1%-5% mannitol, and
c) 3-10 mM acetic acid
wherein the pH is about 3.7.

31. A biologically-active polymer-interferon conjugate composition of claim 1, wherein at least about 65 percent of the antiviral activity is retained relative to native interferon-beta 1b, using the EMC/Vero or EMC/A549 antiviral bioassay.

32. A biologically-active polymer-interferon conjugate composition of claim 1, wherein at least about 20 percent of the antiviral activity is retained relative to native interferon-beta 1b, using the EMC/Vero or EMC/A549 antiviral bioassay.

33. A method of preparing the biologically active polymer-interferon conjugate composition of claim 1, comprising reacting interferon-beta 1b with an activated polyalkylene oxide polymer having a molecular weight of at least about 30 kDa under conditions sufficient to cause conjugation of the activated polyalkylene oxide polymer to the interferon-beta 1b, purifying the resulting conjugate and resuspending the conjugate in a buffered solution having a pH range of about 3.0 to about 8.0, wherein said solution optionally contains an excipient and a surfactant and wherein said composition retains at least about 20% of the antiviral activity is retained relative to native interferon-beta 1b, using the EMC/Vero or EMC/A549 antiviral bioassay.

34. The method of claim 33 wherein the conditions are sufficient to cause conjugation of the activated polyalkylene oxide polymer to the amino-terminal of the interferon-beta 1b.

35. The method of claim 33 wherein the conditions are sufficient to cause conjugation of the activated polyalkylene oxide polymer to an epsilon amino group of a Lys of the interferon-beta 1b.

36. The method of claim 33 wherein the molecular weight of the activated polyalkylene oxide polymer ranges from about 30 kDa to about 40 kDa.

37. The method of claim 33 wherein the molecular weight of the activated polyalkylene oxide polymer is about 30 kDa.

38. The method of claim 33 wherein the molecular weight of the activated polyalkylene polymer is about 40 kDa.

39. The method of claim 33 wherein the activated polyalkylene polymer is an activated polyethylene glycol.

40. The method of claim 39 wherein the activated polyethylene glycol comprises a terminal reactive aldehyde moiety.

41. The method of claim 40 wherein the activated polyethylene glycol is selected from the group consisting of mPEG-CH₂CH₂CH₂CHO, mPEG₂CH₂CH₂CH₂CHO, mPEG-CH₂CH₂CH₂CH₂CHO and mPEG₂-CH₂CH₂CH₂CH₂CHO.

42. The method of claim 39 wherein the activated polyethylene glycol is selected from the group consisting of 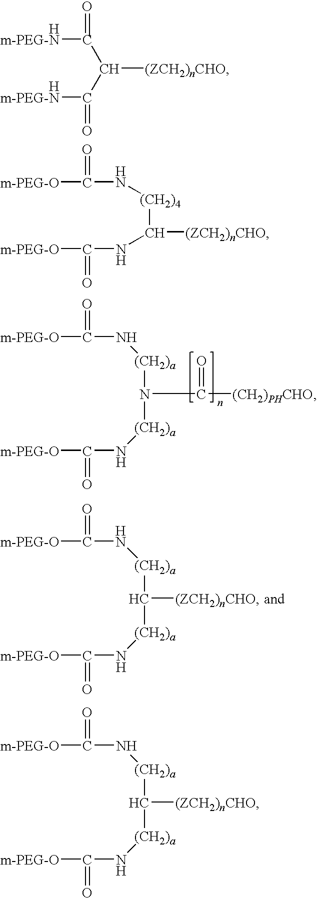
wherein:
(a) is an integer of from about 1 to about 5;
Z is O, NR₈, S, SO or SO₂; where R₈ is H, C₁₋₈ alkyl C₁₋₈ branched alkyl, C₁₋₈ substituted alkyl, aryl or aralkyl;
(x) is the degree of polymerization;
(n) is 0 or 1;
(p) is a positive integer, preferably from about 1 to about 6, and
m-PEG is CH3-O—(CH2CH2O)ₓ—.

43. The method of claim 33, wherein the activated polyethylene glycol comprises a terminal reactive moiety selected from the group consisting of:

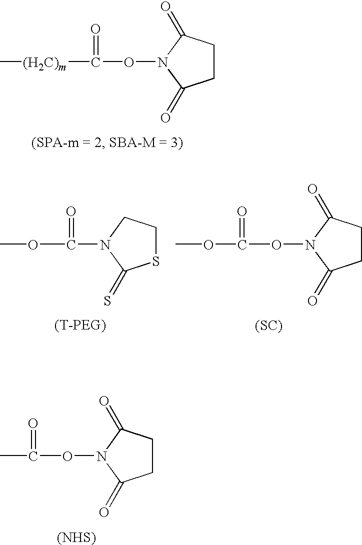

44. A method of administering a composition of claim 1 comprising a first step of neutralizing the acidic buffers followed by administering the composition to a patient in need of such administration.

45. The method of claim 44 wherein the acidic buffer is neutralized with sodium phosphate.

46. The method of claim 44 wherein the composition is administered orally, intravenously, subcutaneously, or intramuscularly.

47. A method of treating a mammal having a disease or disorder responsive to interferon-beta comprising administering an amount of the pharmaceutical composition of claim 1 effective to treat the disease or disorder.

48. A method of preparing a polyalkylene oxide-protein conjugate comprising the steps of
(a) solubilizing a protein of interest in a compatible aqueous solution in the presence of a protein-solubilizing amount of a compatible detergent;
(b) reacting the solubilized protein of interest with an activated polyalkylene oxide polymer, to produce a solution comprising a polyalkylene oxide-protein conjugate and the detergent;
(c) adjusting the reacted solution of step (b) to a pH effective to dissociate the detergent from the polyalkylene oxide-protein conjugate;
(d) separating the dissociated detergent from the polyalkylene oxide-protein conjugate, and recovering the polyalkylene oxide-protein conjugate.

49. The method of claim 48 wherein pH is adjusted in step (c) to a range from about pH 3 to about pH 4.

50. The method of claim 48 wherein the activated polyalkylene oxide polymer is a polyethyelene glycol polymer ranging in size from about 12 kDa to about 60 kDa.

51. The method of claim 48 wherein the detergent is selected from the group consisting of an ionic detergent, a non-ionic detergent, a zwitterionio detergent, and combinations thereof.

52. The method of claim 51 wherein the detergent is a zwitterionic detergent.

53. The method of claim 48 wherein the protein is an interferon.

54. The method of claim 53 wherein the protein is an IFN-beta.


SEQUENCE LISTING (2 sequences)

SEQ ID NO: 1 - protein, 165 aa, Human
  MISC_FEATURE (16)..(16)
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - DNA, 498 bp, Human
atgagttataacctgctgggctttctgcaacgttcttccaattttcaatcgcaaaaactgctgtggcaacttaacgggcgcctggaatattgcttgaaagatcgcatgaactttgacattccggaagaaattaaacagctgcaacagtttcaaaaagaagatgccgcgttgaccatttacgagatgctgcaaaacattttcgccatctttcgccaagattcctccagtacggggtggaacgaaactattgtcgagaatttgctggcgaacgtgtatcaccaaattaatcatttgaaaaccgtgttggaagagaaactggaaaaagaggattttacccggggaaaactgatgtcaagcttgcatctgaaacgttactacggccgtatcctccactacctgaaagccaaagagtatagccactgcgcctggacaattgttcgcgttgaaattctgcgtaacttttattttattaatcgtctcaccggctacctgcgcaat